SEQUENCE LISTING (10 sequences)

SEQ ID NO: 1 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 2 - protein, 265 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 3 - protein, 23 aa, Homo sapiens
AGVIAVIVVVVIAVVAGIVVLVI

SEQ ID NO: 4 - protein, 26 aa, Homo sapiens
SRKKRMAKYEKAEIKEMGEMHRELNA

SEQ ID NO: 5 - DNA, 29 bp, synthetic construct
  misc_feature 1..29: note = forward primer (Oct4 promoter)
  source 1..29: mol_type = other DNA
agcaactggtttgtgaggtgtccggtgac

SEQ ID NO: 6 - DNA, 28 bp, synthetic construct
  misc_feature 1..28: note = reverse primer (Oct4 promoter)
  source 1..28: mol_type = other DNA
ctccccaatcccaccctctagccttgac

SEQ ID NO: 7 - DNA, 30 bp, synthetic construct
  misc_feature 1..30: note = forward primer (Sox2 promoter)
  source 1..30: mol_type = other DNA
ttttcgtttttagggtaaggtactgggaag

SEQ ID NO: 8 - DNA, 30 bp, synthetic construct
  misc_feature 1..30: note = reverse primer (Sox2 promoter)
  source 1..30: mol_type = other DNA
ccacgtgaataatcctatatgcatcacaat

SEQ ID NO: 9 - DNA, 20 bp, synthetic construct
  misc_feature 1..20: note = forward primer (]-actin promoter)
  source 1..20: mol_type = other DNA
aaatgctgcactgtgcggcg

SEQ ID NO: 10 - DNA, 20 bp, synthetic construct
  misc_feature 1..20: note = reverse primer ( ]-actin promoter)
  source 1..20: mol_type = other DNA
aggcaactttcggaacggcg

(19) United States
(12) Patent Application Publication
(10) Pub. No.: US 2026/0125649 A1
(43) Pub. Date: May 7, 2026

(54) MEDIUM COMPOSITION AND METHOD FOR CULTURING MESENCHYMAL STEM CELLS

(71) Applicant: ACADEMIA SINICA, Taipei City (TW)

(72) Inventors: Han-Chung WU, Taipei City (TW); I-I KUAN, Keelung City (TW); Chien-Hsu CHEN, Taichung City (TW)

(73) Assignee: ACADEMIA SINICA, Taipei City (TW)

(21) Appl. No.: 19/439,092

(22) Filed: Jan. 2, 2026

Related U.S. Application Data
(62) Division of application No. 17/273,256.
(60) Provisional application No. 62/726,586, filed on Sep. 4, 2018.

(51) Int. Cl. C12N 5/0775

(52) U.S. Cl. CPC C12N 5/0668; C12N 2500/32; C12N 2500/34; C12N 2500/36; C12N 2500/38; C12N 2500/42; C12N 2500/80; C12N 2501/58

(57) ABSTRACT

The present invention generally relates to a medium composition and method for culturing mesenchymal stem cells (MSCs), in which the medium comprises an epithelial cell adhesion molecule (EpCAM) peptide, particularly a truncated EpCAM polypeptide containing the extracellular domain (EpEX). It significantly enhances cell proliferation and multipotency of the MSCs.

### RELATED APPLICATIONS

This application is a Divisional of application Ser. No. 17/273,256, filed Mar. 3, 2021, which is the National Phase of PCT/US2019/049344, filed Sep. 3, 2019, and which claims the benefit of U.S. provisional application No. 62/726,586, filed Sep. 4, 2018, under 35 U.S.C. § 119. The entire contents of prior applications are hereby incorporated by reference in their entireties.

### REFERENCE TO ELECTRONIC SEQUENCE LISTING

The application contains a Sequence Listing which has been submitted electronically in XML format and is hereby incorporated by reference in its entirety. Said .XML copy, created on Jan. 2, 2026, is named “2026_01_02_Sequence_Listing_5992-0237PUS3.xml” and is 12,563 bytes in size. The sequence listing contained in this .XML file is part of the specification and is hereby incorporated by reference herein in its entirety.

### TECHNOLOGY FIELD

The present invention generally relates to a medium composition and method for culturing mesenchymal stem cells (MSCs), in which the medium comprises an epithelial cell adhesion molecule (EpCAM) peptide, particularly a truncated EpCAM polypeptide containing the extracellular domain (EpEX). It significantly enhances cell proliferation and multipotency of the MSCs. Specifically, the present invention provides a method for enhancing osteogenesis of MSCs by culturing the MSCs under osteogenic conditions in the presence of the EpCAM polypeptide.

### BACKGROUND OF THE INVENTION

MSCs are found in compact bone, tendon, adipose, placenta, and umbilical cord (Brighton and Hunt, 1997), where these cells have the potential to differentiate into multiple lineages, including bone, cartilage, and muscle (Brighton and Hunt, 1997; Valero et al., 2012). In damaged tissues or organs, MSCs secrete chemokines and growth factors to create a microenvironment that promotes repair and recovery, which is especially important in bone regeneration (Briggs and King, 1952). MSC cytokine secretion also modulates the immune system, and because of these varied actions, MSCs are considered to be promising therapeutic candidates with wide-ranging clinical applications. Aging of hMSCs is known to attenuate proliferation, while increasing oxidative damage and senescence (Stolzing et al., 2008; Zhou et al., 2008). Therefore, the use of aged MSCs for autologous cell-based therapies is especially challenging (Stenderup et al., 2003; Stolzing et al., 2008). In addition to reduced proliferation of the MSCs themselves, aging is associated with decreased proliferative capacity in MSC-derived osteo-progenitor cells, which leads to decreased osteoblast cell number and eventually hinders bone formation (Stenderup et al., 2003; Zhou et al., 2008). Because of the diminished proliferative capacity and poor survival, MSCs derived from adult patients currently have limited potential for clinical use. In order to address these obstacles, methods to improve stemness and differentiation of MSCs are now under intensive development.

EpCAM is a type I transmembrane protein with 314 amino acids and a molecular weight of about 39-42 kDa (Litvinov et al., 1994). It contains an extracellular domain (EpEX, 265 amino acids), a single transmembrane domain, and a short intracellular domain (EpICD, 26 amino acids). EpCAM is a well-known tumor-associated antigen, which is enriched in various carcinomas and involved in homotypic cell-cell adhesion in normal epithelium. (Litvinov et al., 1994). Previous research demonstrated that active proliferation is associated with enhanced EpCAM expression in neoplastic tissues. Furthermore, EpCAM is known to be relatively stable within the membrane of normal epithelial tissue, but is prone to cleavage in cancer tissue (Maetzel et al., 2009). Maetzel et al. first shed light on the mechanisms of EpCAM activation, showing that it occurs via regulated intramembrane proteolysis (RIP). During this process, EpCAM is cleaved, generating two products (EpEX and EpICD), which then induce EpCAM-mediated proliferative signaling (Maetzel et al., 2009). After RIP of EpCAM, EpICD associates with FHL2, β-catenin and Lef-1 to form a nuclear complex that binds to DNA at Lef-1 consensus sites and regulates gene transcription, potentially contributing to carcinogenesis.

In a recent study we reported that EpCAM is enriched in human embryonic stem cells (hESCs), where it not only serves as an important surface marker, but it also regulates the four Yamanaka factors (Lu et al., 2010). Similarly, EpCAM plays a critical role in regulating self-renewal, cancer initiating ability, and invasiveness in colon cancer cells (Lin et al., 2012). It is also interesting to note that overexpression of EpCAM or EpICD decreased the levels of p53 and p21, and increased the promoter activity of Oct4 during iPSC derivation (Huang et al., 2011). Based on these findings, we recently further discovered that EpCAM/EpEX, together with Oct4 or Klf4 expression, can generate induced pluripotent stem cells (iPSCs) (Kuan et al., 2017). Despite this growing knowledge about EpCAM function in stem cells, the function of EpCAM/EpEX in human MSCs has not been previously described.

### SUMMARY OF THE INVENTION

In this invention, it is disclosed for the first time that when mesenchymal stem cells (MSCs) are cultured in a medium comprising an EpCAM polypeptide, especially a truncated EpCAM polypeptide containing the extracellular domain (EpEX), the cell proliferation and multipotency of the MSCs are significantly enhanced.

Therefore, in one aspect, the present invention provides a medium composition for culturing MSCs, which comprises a basal medium and an isolated EpCAM polypeptide.

In some embodiments, the EpCAM polypeptide comprises an extracellular domain of EpCAM.

In some embodiments, the EpCAM polypeptide does not include an intracellular domain of EpCAM or a transmembrane domain.

In some embodiments, the EpCAM polypeptide comprises an amino acid sequence at least 90% identical to SEQ ID No: 1.

In some embodiments, the EpCAM polypeptide comprises an amino acid sequence of SEQ ID NO: 1.

In some embodiments, the EpCAM polypeptide is a fragment of EpCAM e.g. an extracellular domain of EpCAM, having an amino acid sequence at least 90% identical to SEQ ID No: 2, preferably SEQ ID NO: 2.

In some embodiments, the EpCAM polypeptide is present in an amount effective in enhancing functional characteristics of MSCs.

In some embodiments, the functional characteristics of MSCs include activities in expansion (proliferation) and/or multipotency (differentiation).

In another aspect, the present invention provides a method for culturing mesenchymal stem cells (MSCs), comprising culturing the MSCs under a condition in the presence of an isolated EpCAM polypeptide. Specifically, the MSCs can be cultured in a medium composition as described herein. Alternatively, MSCs can be cultured in a medium and then an isolated EpCAM polypeptide is added to the medium for further incubation for a proper period of time.

In some embodiments, the MSCs are cultured under a condition that allows proliferation where the medium composition may further include a serum ingredient (for example, fetal bovine serum (FBS)), glutamine, and/or antibiotics (for example, penicillin and streptomycin). In some embodiments, the MSCs are cultured under a condition that allows differentiation of the MSCs toward specific cells of interest where the medium composition may further include certain components for inducing differentiation. In certain examples, to induce osteogenic differentiation, the medium composition is supplemented with a corticosteroid (e.g. dexamethasone), and a phosphate source (e.g. ascorbic acid-phosphate and β-glycerophosphate).

Further provided is use of an isolated EpCAM polypeptide as described herein for manufacturing a reagent (as an activator) for enhancing functional characteristics of MSCs e.g. expansion (proliferation) and/or multipotency (differentiation).

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1. shows that EpEX increases cell proliferation and multipotency factors in mesenchymal stem cells. The proliferation of MSCs was examined by measuring doubling time. MSCs were treated with EpEX (3 μg/mL) for 24 and 48 h. Cell number was counted and then doubling time was calculated. MSCs were treated with EpEX (3 μg/mL) for the indicated times. After treatment, protein expression of cell cycle regulators (cyclin D1, cyclin D2, cyclin D3, cyclin E1, CDK4 and CDK9) and pluripotency factors (Oct4, Sox2, c-Myc, and Lin28) was examined by Western blotting.

FIG. 2A to FIG. 2E show that EpEX upregulates cell cycle regulators and stemness markers via EGFR signaling. FIG. 2A shows that MSCs were treated with EpEX (3 μg/mL) for 15 min and the phosphorylation of EGF receptors was detected by an EGFR phosphorylation antibody array. FIG. 2B shows that MSCs were treated with EpEX (3 μg/mL) for the indicated times. Phospho-EGFR (Tyr845) was detected by Western blotting. FIG. 2C shows that MSCs were pretreated with or without an EGFR inhibitor (AG1478, 25 μM) for 30 min and then cells were incubated with EpEX for 18 h. After treatment, cell cycle progression was investigated by flow cytometry with PI staining. Fraction of cells in each phase (G1, S, G2/M) of the cell cycle was evaluated. Cells that expressed EGFR shRNA or shLuc were treated with EpEX for 18 h. After treatment, cell cycle progression was investigated by flow cytometry with PI staining. Fraction of cells in each phase (G1, S, G2/M) of the cell cycle was evaluated. FIG. 2D shows that MSCs were pretreated with or without AG1478 and then stimulated by EpEX. The protein expression of cell cycle regulators (cyclin D1, cyclin D2, cyclin E1) and pluripotency factors (Oct4, Sox2, c-Myc, Lin28) was examined by Western blotting. MSCs expressing EGFR shRNA were stimulated by EpEX. After treatment, the protein expression of cell cycle regulators (cyclin D1, cyclin E1, CDK4 and CDK9) and pluripotency factors (Oct4, in Sox2, c-Myc, and Lin 28) was examined by Western blotting. FIG. 2E shows that MSCs expressing EGFR shRNA were stimulated with EpEX. After treatment, the gene expression of pluripotency factors (Oct4, Sox2, c-Myc, Lin28 and EpCAM) was examined by qPCR.

FIG. 3A to FIG. 3E shows that EpEX upregulates cell cycle regulators and stemness markers via EGFR-STAT3 signaling. FIG. 3A shows that MSCs were treated with EpEX (3 μg/mL) for the indicated times. After treatment, the protein levels of total STAT3 and phospho-STAT3 (Tyr705) were examined by Western blotting. FIG. 3B shows that MSCs were treated with EGFR shRNA or shLuc, and total STAT3 and phospho-STAT3 (Tyr705) were examined by Western blotting with or without EpEX treatment. FIG. 3C shows that cells were pretreated with a STAT3 inhibitor (WP1066, 5 μM), followed by stimulation with EpEX for 18 h. Cell cycle progression was investigated by flow cytometry with PI staining. Fraction of cells in each phase (G1, S, G2/M) of the cell cycle was evaluated. Cells expressing STAT3 shRNA were treated with EpEX for 18 h, after which cell cycle progression was investigated by flow cytometry with PI staining. Fraction of cells in each phase (G1, S, G2/M) of the cell cycle was evaluated. FIG. 3D shows that MSCs were pretreated with or without WP1066 and then stimulated by EpEX. The protein levels of cell cycle regulators (cyclin D1, cyclin D2, cyclin E1, CDK4 and CDK9) and pluripotency factors (Oct4, Sox2, c-Myc, Lin28) were examined by Western blotting. MSCs expressing STAT3 shRNA or shLuc were stimulated with EpEX. After treatment, the protein levels of cell cycle regulators (cyclin D1, cyclin D2, cyclin E1, CDK4 and CDK9) and pluripotency factors (Oct4, Sox2, c-Myc, and Lin 28) were examined by Western blotting. FIG. 3E shows that MSCs expressing STAT3 shRNA or shLuc were stimulated with EpEX. After treatment, the gene expression of pluripotency factors (Oct4, Sox2, c-Myc, Lin28 and EpCAM) was examined by qPCR.

FIG. 4A to FIG. 4B show that EpEX suppresses miRNA, let-7, through EGFR-STAT3-Lin28 signaling. FIG. 4A shows that Cells were treated with EpEX (3 μg/mL), and the expression of let-7 was detected by qPCR. MSCs were pretreated with or without AG1478, or WP1066, and then stimulated with EpEX. The expression of let-7 was detected by qPCR. MSCs expressing EGFR shRNA, STAT3 shRNA or Lin28b shRNA were stimulated with EpEX. The expression of let-7 was detected by qPCR. FIG. 4B shows that MSCs were transfected with a let-7 inhibitor or a let-7 mimetic, and then stimulated with EpEX. Expression of pluripotency factors (Oct4, Sox2, c-Myc and Lin28) was examined by qPCR. MSCs were transfected with a let-7 mimetic, and then stimulated with EpEX. Protein levels of pluripotency factors (Oct4, Sox2, c-Myc, and Lin28) were examined by Western blotting.

FIG. 5A to FIG. 5C show that EpEX upregulates HMGA2 and increases its binding to the promoters of Oct4 and Sox2 through EGFR-STAT3-Lin28-let-7 signaling. FIG. 5A shows that MSCs were treated with EpEX (3 μg/mL) for the indicated times, and expression of HMGA2 was detected by Western blotting. MSCs were treated with EpEX (3 μg/mL), and the expression of HMGA2 was detected by immunofluorescence staining. FIG. 5B shows that MSCs were treated with let-7 mimetic or let-7 inhibitor, followed by the treatment with EpEX (3 μg/mL) for 12 h. To detect the binding of HMGA2 to Oct4 promoters, cross-linked DNA was isolated and then amplified with specific primers by qPCR. FIG. 5C shows that MSCs were treated with let-7 mimetic, followed by the treatment with EpEX (3 μg/mL) for 12 h. The gene expression of HMGA2 is detected by qPCR. MSCs were treated with let-7 mimetic and then treated with EpEX (3 μg/mL) for 12 h. The protein abundance of HMGA2 was examined by Western blotting.

FIG. 6A to FIG. 6D show that EpEX enhances MSC bone formation by upregulating RUNX2. FIG. 6A shows that MSCs were treated with EpEX for 14 days during osteo-induction. Calcium precipitation was measured by Alizarin Red S (ARS) staining to probe the efficiency of osteogenesis. This method shows higher calcium precipitation in EpEX (Day 14) treated cells than non-treated controls. Quantification of osteogenesis, as measured by ARS staining, is shown for each group. MSCs were induced by osteogenetic medium and treated with EpEX at indicated doses. The gene expression of RUNX2 was examined by qPCR. FIG. 6B shows that MSCs were pretreated with let-7 mimetic and then treated with EpEX for 14 days during osteo-induction. ARS staining was performed to check the efficiency of osteogenesis. MSCs were pretreated with let-7 inhibitor and then treated with EpEX for 14 days during osteo-induction. ARS staining was performed to check the efficiency of osteogenesis. FIG. 6C shows that MSCs were pretreated with let-7 mimetic and then induced by EpEX. RUNX2 gene expression was measured by qPCR. MSCs were pretreated with let-7 inhibitor and induced by EpEX. RUNX2 gene expression was examined by qPCR. FIG. 6D shows a schematic showing the functional roles of EpCAM/EpEX in MSCs. Upon EpEX stimulation, phosphorylation of EGFR-STAT3 signaling is induced and subsequently upregulates the level of Lin28 which inhibits let-7. When let-7 is inhibited, the transcription factor, HMGA2, is increased and binds to the promoters of Oct4 and Sox2. The EpEX-mediated increases of Oct4 and Sox2 can promote osteogenesis of MSCs during osteo-induction.

FIG. 7. The effect of EpEX on the phosphorylation of protein kinase receptor. MSCs were treated with EpEX (3 μg/mL) for the indicated times. After treatment, cells were harvested and the phosphorylation of protein kinase receptors was detected by an RTK membrane array. The phosphorylation level of EpEX-treated cells was normalized to non-treated control cells. The spots corresponding to quantification results are indicated by the numbers 1-5.

FIG. 8. EpEX and EGF induce the phosphorylation of EGFR and STAT3. MSCs were pretreated with or without an EGFR inhibitor, AG1478, and followed by the treatment with either EGF, EpEX, or co-treated with EGF and EpEX at indicated time. The phosphorylation of EGFR (Tyr845) and STAT3 (Tyr705) were examined by Western blotting with specific antibodies.

FIG. 9. EpEX induces the phosphorylation and activity of TACE and γ-secretase. MSCs were stimulated by EpEX for the indicated times, and the activity of TACE and γ-secretase were detected. MSCs were stimulated by EpEX (3 μg/mL) for the indicated times. Western blot analysis was performed to detect the phosphorylation of TACE and Presenilin 2.

FIG. 10. EpEX and EGF induce the phosphorylation of TACE, ERK1/2 and PS2. MSCs were treated with either EGF, EpEX, or co-treated with EGF and EpEX for 5 min. The phosphorylation of ERK1/2 was examined by Western blotting with specific antibodies. MSCs were pretreated with or without an EGFR inhibitor, AG1478, followed by the treatment with either EGF, EpEX, or co-treated with EGF and EpEX for indicated times. The phosphorylation of ERK1/2, TACE (Ser435), PS2 (Ser327) were examined by Western blotting with specific antibodies.

FIG. 11. TACE and presenilin 2 are crucial for the expression of cell cycle regulators and pluripotent markers. In MSCs, TACE was knocked down and the levels of cell cycle regulators and pluripotency markers were examined by Western blotting with specific antibodies. In MSCs, presenilin 2 was knocked down and the levels of cell cycle regulators and pluripotency markers were examined by Western blotting with specific antibodies.

FIG. 12. EpEX increases the binding of EpICD to the promoter of Oct4 by inhibiting let7. (A) MSCs were transfected with let7 inhibitor or mimetics then treated with EpEX (3 μg/mL). Binding of EpICD to the Oct4 promoter was examined by chromatin immunoprecipitation (ChIP). EpICD was pulled down by a specific anti-EpICD antibody. The cross-linked DNA was isolated and then probed by qPCR with specific primers for the Oct4 promoter. (B) MSCs were transfected with let7 inhibitor or mimetics then treated with EpEX (3 μg/mL). Binding of EpICD-HMGA2 was examined by sequential ChIP. EpICD was pulled down by a specific anti-EpICD antibody, followed by pull-down with a HMGA2 antibody. To detect bound Oct4 promoter, the cross-linked DNA was isolated and then amplified by qPCR with specific primers.

FIG. 13. EpCAM is crucial for maintaining expression of cell cycle regulators and stemness markers. The inhibition of EpCAM significantly decreases phospho-STAT3, cell cycle regulators, and stemness markers. MSCs were made to express EpCAM shRNA, and the phosphorylation of STAT3 and total STAT3 were detected by Western blotting. The protein levels of pluripotency factors (Sox2, Oct4, c-Myc and EpCAM) and cell cycle regulators (cyclin D and CDK4) were detected by Western blotting. The level of let-7 was detected by qPCR.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, “mesenchymal stromal/stem cells (MSCs)” can self-renew and are multipotent. The term “multipotency” herein refers to a stem cell that has the ability to differentiate into more than one cell types. Multipotent stem cells cannot give rise to any type of mature cells in the body; they are restricted to a limited range of cell types. For example, MSCs can differentiate into osteoblasts, adipocytes, chondrocytes, neurons, β islet cells, intestine cells. MSCs can be obtained from various sources, such as bone marrow (BMMSCs), adipose or dental tissues and then cultured for expansion.

As used herein, the term “proliferation” or “expansion” can refer to growth and division of cells. In some embodiments, the term “proliferation” or “expansion” as used herein with respect to cells refers to a group of cells that can increase in number over a period of time.

As used herein, the term “polypeptide” or “peptide” refers to a polymer composed of amino acid residues linked via peptide bonds. For example, a polypeptide or a peptide can be a polymer composed of linked amino acids e.g. 500 amino acids or less, e.g. 400 or less, 300 or less, 250 or less, 200 or less, 150 or less, 125 or less, 100 or less, 90 or less, 80 or less, 70 or less, 60 or less, 50 or less or 40 or less amino acids in length.

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of #10% around the cited value.

As used herein, “corresponding to,” refers to a residue at the enumerated position in a protein or peptide, or a residue that is analogous, homologous, or equivalent to an enumerated residue in a protein or peptide.

As used herein, the term “substantially identical” refers to two sequences having more than 85%, preferably 90%, more preferably 95%, and most preferably 100% homology.

As used herein, the term “EpCAM” generally refers to a full-length epithelial cell adhesion molecule (EpCAM). Specifically, EpCAM can include the amino acid sequence set forth in SEQ ID NO: 1 (human EpCAM, corresponds to UniProtKB-P16422). It comprises an extracellular domain, referred to herein as “EpEX”, which is 265 amino acids in length (SEQ ID NO: 2) (i.e. amino acids 1-265 in SEQ ID NO: 1), a single transmembrane domain which is 23 amino acids in length (SEQ ID NO: 3) (i.e. amino acids 266-288 in SEQ ID NO.: 1), and an intracellular domain, referred to herein as ‘Ep-ICD”, which is 26 amino acids in length (SEQ ID NO: 4) (i.e. amino acids 289-314 in SEQ ID NO. 1). A full-length EpCAM can also include those comprising an amino acid sequence which (i) are substantially identical to the amino acid sequences set forth in SEQ ID NO: 1 (for example, at least 85% (e.g., at least 90%, 95% or 97%) identical to SEQ ID NO: 1); and (ii) are encoded by a nucleic acid sequence capable of hybridizing under at least moderately stringent conditions to any nucleic acid sequence encoding the EpCAM set forth herein or capable of hybridizing under at least moderately stringent conditions to any nucleic acid sequence encoding the EpCAM set forth herein, but for the use of synonymous codons (e.g. a codon which does not have the identical nucleotide sequence, but which encodes the identical amino acid). EpCAM as described herein includes human EpCAM and its homologues from vertebrates, and particularly those homologues from mammals.

As used herein, the term “an EpCAM polypeptide” includes a full-length EpCAM or a naturally or non-naturally occurring truncated fragment derived therefrom or functional variants thereof. In some embodiments, an EpCAM polypeptide as described herein may lack the single transmembrane domain and the intracellular domain. For example, such EpCAM polypeptide contains the extracellular domain, without the single transmembrane domain and the intracellular domain, or consists of the extracellular domain only. In particular examples, an EpCAM polypeptide includes an amino acid sequence set forth in SEQ ID NO: 2, or an amino acid sequence at least 85% (e.g., at least 90%, 95% or 97%) identical to SEQ ID NO: 2.

In some instances, any of the EpCAM polypeptide described herein may have up to 500 amino acid in length, for example, containing about 450 amino acid residues, about 350 amino acid residues, about 300 amino acid residues, about 280 amino acid residues, about 275 amino acid residues, about 270 amino acid residues, or about 265 amino acid residues.

To determine the percent identity of two amino acid sequences, the sequences are aligned for optimal comparison purposes (e.g., gaps can be introduced in the sequence of a first amino acid sequence for optimal alignment with a second amino acid sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

It is understandable that a polypeptide may have a limited number of changes or modifications that may be made within a certain portion of the polypeptide irrelevant to its activity or function and still result in a variant with an acceptable level of equivalent or similar biological activity or function. The term “acceptable level” can mean at least 20%, 50%, 60%, 70%, 80%, or 90% of the level of the referenced protein as tested in a standard assay as known in the art. Biologically functional variant polypeptides are thus defined herein as those polypeptides in which certain amino acid residues may be substituted. Polypeptides with different substitutions may be made and used in accordance with the invention. Modifications and changes may be made in the structure of such polypeptides and still obtain a molecule having similar or desirable characteristics. For example, certain amino acids may be substituted for other amino acids in the peptide/polypeptide structure without appreciable loss of activity. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The polypeptide of the present invention may be produced by chemical synthesis using techniques well known in the chemistry of proteins such as solid phase synthesis or synthesis in homogenous solution.

Alternatively, the polypeptide of the present invention may be prepared using recombinant techniques. In this regard, a recombinant nucleic acid comprising a nucleotide sequence encoding a polypeptide of the present invention and host cells comprising such recombinant nucleic acid are provided. The host cells may be cultured under suitable conditions for expression of the polypeptide of interest. Expression of the polypeptides may be constitutive such that they are continually produced or inducible, requiring a stimulus to initiate expression. In the case of inducible expression, protein production can be initiated when desired by, for example, addition of an inducer substance to the culture medium, for example, isopropyl β-D-1-thiogalactopyranoside (IPTG) or methanol. Polypeptide can be recovered and purified from host cells by a number of techniques known in the art, for example, chromatography e.g., HPLC or affinity columns.

The term “polynucleotide” or “nucleic acid” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

The term “encoding” refers to the inherent property of specific sequences of nucleotides in a polynucleotide (e.g., a gene, a cDNA, or an mRNA) to serve as templates for synthesis of other polymers and macromolecules in biological processes having either a defined sequence of nucleotides (i.e., rRNA, tRNA and mRNA) or a defined sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above mentioned purposes. A “recombinant cell” refers to a host cell that has had introduced into it a recombinant nucleic acid. “Transformation” refers to a genetic change in a cell following incorporation of new DNA (i.e., DNA exogenous to the cell). “Transfection” means the transformation of a cell with DNA from a virus. “A transformed cell” mean a cell into which has been introduced, by means of recombinant DNA techniques, a DNA molecule encoding a protein of interest.

Vectors may be of various types, including plasmids, cosmids, fosmids, episomes, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprises, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, an operator sequence, a secretion signal sequence (e.g., □-mating factor signal) and other control sequence (e.g., Shine-Dalgarno sequences and termination sequences). Preferably, vectors may further contain a marker sequence (e.g., an antibiotic resistant marker sequence) for the subsequent screening procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes, but is not limited to, a His-tag fused polypeptide and a GST fused polypeptide. Therefore, in some embodiments, the polypeptide of the invention as described herein can be a fused polypeptide with a tag for purification.

In some embodiments, the polypeptide of the present invention can be said to be “isolated” or “purified” if it is substantially free of cellular material or chemical precursors or other chemicals that may be involved in the process of peptide preparation. It is understood that the term “isolated” or “purified” does not necessarily reflect the extent to which the polypeptide has been “absolutely” isolated or purified e.g. by removing all other substance s (e.g., impurities or cellular components). In some cases, for example, an isolated or purified polypeptide includes a preparation containing the peptide having less than 50%, 40%, 30%, 20% or 10% (by weight) of other proteins (e.g. cellular proteins), having less than 50%, 40%, 30%, 20% or 10% (by volume) of culture medium, or having less than 50%, 40%, 30%, 20% or 10% (by weight) of chemical precursors or other chemicals involved in synthesis procedures.

According to the present invention, an EpCAM polypeptide is added to a culture medium for culturing MSCs. An EpCAM polypeptide can act as an enhancer or activator for promoting functional characteristics of MSCs during the culture.

The terms “culture medium” and “medium” refer to any medium in which animal cells can be cultured. A “basal medium” can refer to a culture medium that contain essential ingredients useful for cell growth including a carbon source, nitrogen source, inorganic salts, and the like, for instance amino acids, lipids, carbon source, vitamins and mineral salts. Examples of commercially available basal media include minimal essential medium (MEM) such as Eagle's medium, Dulbecco's modified Eagle's medium (DMEM), minimum essential medium a (MEM-α), mesenchymal cell basal medium (MSCBM), Ham's F-12 and F-10 medium, DMEM/F12 medium. A culture medium can be free of proteins and/or free of serum, and/or can be supplemented by additional ingredients such as amino acids, salts, sugars, vitamins, hormones, growth factors, depending on the needs of the cells in culture. In some instances, a culture medium may contain serum, at a concentration ranging from 5% to 25%, particularly 10% to 20%. Culture medium for use in proliferation or differentiation of MSCs into specific cells of interest can be available in this art.

Specifically, a culture medium contains an EpCAM polypeptide as described herein in an amount effective in enhancing functional characteristics of MSCs. Said functional characteristics include for example the activities in expansion/proliferation and/or multipotency/differentiation of MSCs. The enhancement of MSCs' functional characteristics can be determined by methods known in the art e.g. based on increase of expression of representative MSC markers, e.g. Oct4, Sox2, c-Myc and Lin28, a reduced doubling time, and differentiation activity assays. In some instances, an EpCAM polypeptide is present in the medium in a concentration of at least about 1 μg/mL, e.g. 3 μg/mL or more, 5 μg/mL or more, 10 μg/mL or more, 25 μg/mL or more, 50 g/mL or more. In some instances, an EpCAM polypeptide is present in the medium in a concentration of 1-50 μg/mL, e.g. 1-25 μg/mL, 1-10 μg/mL, or 1-5 μg/mL.

In some embodiments, a medium composition according to the present invention is provided for culturing MSCs for expansion, which may include a basal medium, a serum ingredient (for example, fetal bovine serum (FBS)), glutamine, and/or antibiotics (for example, penicillin and streptomycin). In some examples, the medium composition may contain a basal medium e.g. Dulbecco's modified Eagle's medium (DMEM e.g. low glucose), supplemented with 5% to 25% FBS, 0.1-5 mM glutamine, and 1-50 μg/mL EpCAM polypeptide.

In some embodiments, a medium composition according to the present invention is provided for culturing MSCs for differentiation. In some instances, to induce osteogenic differentiation, the medium composition may include a basal medium, a serum ingredient (for example, fetal bovine serum (FBS)), a corticosteroid (e.g. dexamethasone), and a phosphate source (e.g. ascorbic acid-phosphate and β-glycerophosphate). In some examples, the medium composition may contain a basal medium e.g. Dulbecco's modified Eagle's medium (DMEM e.g. high glucose), supplemented with 5% to 25% FBS, 0.05-0.5 μM dexamethasone (a corticosteroid), 1-50 mM β-glycerophosphate and 0.01-0.1 mM ascorbic acid-phosphate (a phosphate source), and 1-50 μg/mL EpCAM polypeptide.

According to the present invention, MSCs are cultured under a condition in the presence of an isolated EpCAM polypeptide. Specifically, the MSCs can be cultured in a medium composition as described herein, or MSCs can be cultured in a medium and then an isolated EpCAM polypeptide is added to the medium for incubation for a proper period of time. In some embodiments, MSCs are cultured in a 5% CO₂ incubator at 37° C. In some embodiments, the cell culture can be carried out for at least 1 day or more, 2 days or more, 3 days or more, 4 days or more, 5 days or more, 7 days or more, 14 days or more, 21 days or more, 28 days or more, as needed. In some embodiments, the cells are exposed (or treated) with an isolated EpCAM polypeptide as described herein for a period of time sufficient for enhancing functional characteristics of MSCs. In some embodiments, the duration of exposure (or treatment) with the EpCAM polypeptide is 15 min or more, e.g. 30 min or more, 60 min or more, 120 min or more, 3 hours or more, 6 hours or more, 18 hours or more, 24 hours or more, 48 hours or more, 3 days or more, 4 days or more, 5 days or more, 7 days or more, 14 days or more, 21 days or more, 28 days or more.

The method of the present invention can further include steps to perform routine assays to confirm one or more features of the MSCs after culture, for example, electron microscope, immunological staining and flow cytometer. A cell marker detection can be used to confirm the enhanced level of functional characteristics of the MSCs.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

The main purpose for our current study was to investigate whether EpCAM signaling can promote multipotency and increase cell proliferation in MSCs. Herein, we not only describe a novel molecular mechanism for the regulation of self-renewal in MSCs through EGFR-STAT3 signaling, but we also provide a new method for maintaining multipotency of MSCs that may be useful to advance research into regenerative medicine.

### 1. Material and Methods

### 1.1 Cell Culture

All experiments with primary human cells were conducted in accordance with relevant guidelines and regulations. Human primary bone marrow mesenchymal stem cells (BMMSCs) were purchased from LONZA and were cultured with Dulbecco's Modified Eagle Media-low glucose (DMEM-LG) medium containing 16.6% FBS, 1 mM L-glutamine (Invitrogen), 100 μg/ml Penicillin/Streptomycin (Gibco). All cells were cultured at 37° C. and 5% CO₂. All experiments on primary cells were performed within 10 passages.

### 1.2 Plasmids and Lentivirus Preparation

For knockdown experiments, human EGFR, EpCAM, STAT3 and Lin28 shRNAs in the pLKO vector were obtained from RNAi core facility (Academia Sinica, Taipei). Lentivirus was produced according to standard protocols with minor modifications. In brief, 293T cells were seeded at a density of 70% in a 100-mm dish and transfected with packaging vectors (pCMV-ΔR8.91, containing gag, pol and rev genes), envelope vectors (pMD2.G; VSV-G expressing plasmid), and an individual shRNA vector. The shRNA plasmids were transfected into 293T cells by poly-jet transfection reagent (SignaGen Laboratories). After overnight incubation, the medium was changed to BSA-containing media. MSCs were infected with viral supernatant, containing polybrene (8 μg/ml), for 24 h. The infection procedure was repeated, and cells were incubated in puromycin (2 μg/ml) for 7 days to select cells with stable shRNA expression.

### 1.3 Osteogenic Differentiation

Human primary BMMSCs were cultured in DMEM-LG medium with 10% FBS. Fibroblasts were cultured in DMEM-HG with 10% FBS. To induce differentiation, cells (1×10⁴ cells/cm²) were cultured with osteogenic induction medium (90% DMEM-HG, 10% FBS, 0.1 μM dexamethasone, 10 mM β-glycerophosphate, and 0.05 mM L-ascorbic acid phosphate). The media was replaced twice per week during the differentiation period.

### 1.4 Alizarin Red S Staining

After 14 days of osteogenic differentiation, cells were fixed with ice-cold 70% ethanol at −20° C. for 1 h and then washed with PBS. The cells were then stained with 40 mM Alizarin Red S (ARS) (pH 4.2) for 10 min and subsequently washed five times with ddH₂O before being air dried. For quantification, the cells were incubated with 1 mL of acetyl pyridinium chloride buffer for 1 h to extract ARS, and the O.D. at 550 nm was recorded.

### 1.5 Quantitative Real Time RT-PCR

Total RNA was extracted using TRI reagent (Invitrogen, CA, USA), and 5 μg of total RNA was reverse transcribed using oligo (dT) primer (Fermentas, Glen Burnie, MD, USA) with SuperScript III reverse transcriptase (Invitrogen). Quantitative real time RT-PCR (qPCR) was performed on cDNA using the Light Cycler 480 SYBR Green I Master kit (Roche Applied Science, Indianapolis, IN) and the LightCycler480 System (Roche Applied Science). The gene expression levels of each sample were normalized to the expression levels of glyceraldehyde 3-phosphate dehydrogenase (GAPDH).

### 1.6 Western Blot Analysis and Phospho-Kinase Array

Western blotting was performed as previously described (Takahashi et al., 2003). Cells were lysed in lysis buffer (150 mM NaCl, 50 mM Tris-HCl (pH 7.4), 1% Nonidet P-40), containing a protease inhibitor mix (Roche Applied Science). Nuclear fractions and cytoplasmic fractions were separated by the Nuclear/Cytosol Fractionation Kit according to the manufacturer's instructions (BioVision Inc., Milpitas, CA, USA). Protein samples were separated by SDS-PAGE under denaturing conditions, and transferred to a PVDF membrane (Millipore). To probe for pluripotency markers, membranes were incubated with the indicated antibodies against Oct4 (1:1000, Abcam, Cambridge, UK), Nanog (1:1000, Genetex), Lin28 (1:1000, Genetex) or Sox2 (1:1000, Genetex). The CDK and cyclin antibodies were from the CDK and Cyclin Antibody Sampler Kits (Cell Signalling Technology, #9868 and #9869 respectively), including antibodies against cylin D1, D2, E1, and CDK4, CDK9 (1:1000). EpCAM (1:1000, Genetex), phospho-EGFR (1:1000, Cell signaling), EGFR (1:1000, Cell signaling), HMGA2 (1:1000, Cell signaling) or GAPDH (1:10000, Abcam) were also used. After incubation with primary antibody, the membranes were incubated with horseradish peroxidase (HRP)-conjugated secondary antibodies, goat anti-mouse IgG (1:3000, Santa Cruz, CA) or goat anti-rabbit IgG (1:3000, Santa Cruz, CA). Finally, membranes were washed three more times, and developed using Chemiluminescence Reagent Plus (Thermo Fisher Scientific, Runcorn, UK). The Phospho-Kinase Array Kit (Proteome Profiler Antibody Array, R&D Systems) was used according to the manufacturer's instructions.

### 1.7 Flow Cytometry Analysis

Cells were dissociated with 0.25% trypsin-EDTA (1 mM) (Invitrogen) for 3 min, washed with fluorescence-activated cell sorting buffer (FACS buffer, PBS containing 1% fetal bovine serum), fixed in 4% PFA, and then permeabilized with 0.1% Triton X-100 in PBS. Subsequently, cells were stained with Oct4, Sox2 or Nanog antibodies (1:100, ab 107156, Abcam, UK), washed and suspended in FACS buffer, and incubated with secondary antibody (1:200, Jackson ImmunoResearch) for 60 min at room temperature. Flow cytometry analysis was performed with a BD FACSCanto II flow cytometer (BD Biosciences, CA, USA).

### 1.8 Immunofluorescence Staining

MSCs were seeded onto Millicell EZ slides (Millipore), and then iPSCs or ESCs were seeded. Cells were washed, fixed in 4% PFA for 10 min, and then permeabilized with 0.1% Triton X-100 for 10 min. Cells were stained with HMGA2 antibody (1:1000, Cell Signaling) for 60 min at room temperature, and then washed with PBS. Then the slides were incubated with goat anti-rabbit antibody conjugated with Alexa Fluor 568 (1:250; Invitrogen) for 1 h. After washing, the nuclei were stained with 4′, 6-diamidino-2-phenylindole (DAPI) (1:1000) (Invitrogen). Cells were observed by confocal microscopy (TCS SP5; Leica, Wetzlar, Germany).

### 1.9 Chromatin Immunoprecipitation

We performed chromatin immunoprecipitation (ChIP) with the Pierce™ Magnetic ChIP Kit (Thermo Fisher Scientific), to according the manufacturer's instructions. In brief, the protein-DNA complexes were cross-linked with 1% formaldehyde and quenched by adding glycine to a final concentration of 200 mM. The chromatin complexes were sonicated to an average size of 250 bp by a MISONIX Sonicator 3000. For immunoprecipitation, 4 μg of anti-HIF2 (Novus) was incubated with protein G beads (Invitrogen) for 4 h. The immunocomplexes were further incubated with chromatin for another 4 h. The bound fraction was isolated by protein G beads according to the manufacturer's instructions, and the immunocomplexes were subjected to reverse cross-linking. In double ChIP analysis, sequential (double) immunoprecipitation of two chromatin-binding proteins was performed to detect co-occupancy of proteins on promoter regions of pluripotency genes. We followed a previously described protocol (Peng and Chen, 2013). Briefly, we performed the first-round ChIP by using the anti-HMGA2 antibody (Cell Signaling Technologies). The cross-linked DNA-protein complex was washed and eluted with 10 mM dithiothreitol (DTT) at 37° C. for 1 h. The eluents were then diluted 50-fold in a ChIP buffer (0.01% SDS, 1.1% TX-100, 1.2 mM EDTA, 16.7 mM Tris-HCl pH 8.1, 167 mM NaCl). A second-round of ChIP was performed with anti-HIF2 (Novus) or the control IgG antibody (Thermo Fisher Scientific). Chromatin was collected from the protein G-agarose beads after washing by elution with sodium bicarbonate-SDS buffer.

The immunoprecipitated DNA was recovered by a PCR purification kit (Thermo Fisher Scientific), and the purified DNA was subjected to real time quantitative PCR for further analysis. Immunoprecipitation/input was calculated for each gene and each gene was further normalized to the level of mouse β-actin promoter. The following primers were used: Oct4 promoter, forward: 5′-AGCAACTGGTTTGTGAGGTGTCCGGTGAC-3′ (SEQ ID NO: 5), and reverse: 5′-CTCCCCAAT CCCACCCTCTAGCCT TGAC-3′ (SEQ ID NO: 6), Sox2 promoter, forward: 5′-TTTTCGTTTTTAGGGTAAGGTACTGGGAAG-3′ (SEQ ID NO: 7), and reverse: 5′-CCACGTGAATAATCCTATATGCATCACAAT′ (SEQ ID NO: 8); and β-actin promoter: forward: 5′-AAATGCTGCACTGTGCGGCG-3′ (SEQ ID NO: 9), and reverse: 5′-AGGCAACTTTCGGAACGGCG-3′ (SEQ ID NO: 10) (Hattori et al., 2004).

### 1.10 TACE Activity and γ-Secretase Activity Assay

ADAM17 activity was measured using the InnoZyme ADAM17 activity kit (Calbiochem). In brief, cell lysates were harvested and loaded into a TACE antibody-coated plate. After 1 h incubation, the lysate was removed and the plate was washed twice. Substrate was added into each well for 5 h at 37° C. After incubation, the fluorescence signal of the reaction product was detected at excitation of 324 nm and emission of 405 nm. For the detection of γ-secretase activity, cell lysates were extracted and 500 μg protein was used. γ-secretase activity was detected by γ-secretase substrate (35 μM).

### 1.11 Statistical Analysis

All data are presented as mean±SEM for the indicated number of experiments. Unpaired Student's t-test was performed to calculate the statistical significance of the expression percentages versus those of control cultures. A p-value of less than 0.05 was considered statistically significant.

### 2. Results

### 2.1 EpEX Enhances Cell Proliferation and Self-Renewal in Mesenchymal Stem Cells

A recent study showed that CD49f increases growth of MSCs and sustains multipotency via the regulatory effects on Oct4 and Sox2 (Yu et al., 2012). We have previously defined EpCAM as a critical stem cell marker, and showed that EpICD can regulate Oct4 and Sox2 gene expression by binding to their promoters (Lu et al., 2010). We also recently reported that EpCAM/EpEX cooperates with Oct4 or Klf4 to induce iPSC formation from mouse embryonic fibroblasts, and discovered a novel mechanism through which EpCAM/EpEX regulates STAT3-HIF2α signaling (Kuan et al., 2017). Based on these previous reports, we suspected that EpEX may play a role in helping MSCs to maintain pluripotency.

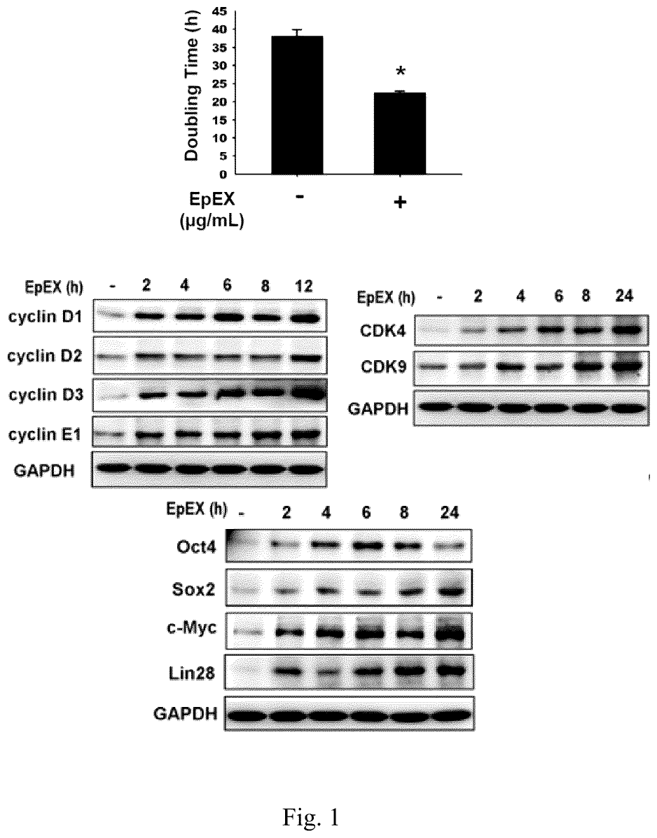

We used human bone marrow-derived MSCs to study the effects of EpEX and first investigated whether EpEX promotes cell proliferation of MSCs. Interestingly, we found that EpEX shortened the doubling time of MSCs from 38.2 h to 22.5 h (FIG. 1, Table 1). Next, we examined the effect of EpEX on cell cycle progression by flow cytometry with propidium iodide (PI) staining. We showed that EpEX increased the percentage of cells in G2/M phase from 6.5% to 29.3% at 18 hdata not shown. EpCAM has been reported to enhance cell cycle progression through upregulation of the proto-oncogene c-Myc and cyclin A/E (Munz et al., 2004). Additionally, EpCAM is known to upregulate cyclin D1 via its direct interaction partner, FHL2, and downstream events such as phosphorylation of the retinoblastoma protein, Rb (Chaves-Perez et al., 2013). Therefore, we further asked whether EpEX can function to upregulate the expression of cell cycle regulators and pluripotency markers. We first performed Western blotting and found that EpEX significantly increased the protein expression of cell cycle regulators, including cyclin A2, cyclin D1, cyclin D2, cyclin D3 and cyclin E1, as well as CDK4 and CDK9 (FIG. 1). Surprisingly, EpEX also significantly increased the protein expression of pluripotency markers, including Oct4, Sox2, c-Myc and Lin28 (FIG. 1). We then used flow cytometry to confirm that EpEX increased the protein levels of the stemness markers, Oct4, Sox2, c-Myc and EpCAM, and qPCR to probe mRNA expression levels (data not shown). From these experiments, we found that EpEX accelerates MSC proliferation and enhances expression of multipotency markers.

TABLE 1
The effect of EpCAM and EpEX on MSC doubling time

| EpEX (μg/mL ) | 0 | 3 |
|---|---|---|
| P3 | 17.6 ± 0.3 h | 16.1 ± 0.1 h |
| P9 | 38.2 ± 1.7 h | 22.5 ± 0.4 h |

### 2.2 EpEX Induces Cell Proliferation and Self-Renewal Through EGFR Signaling

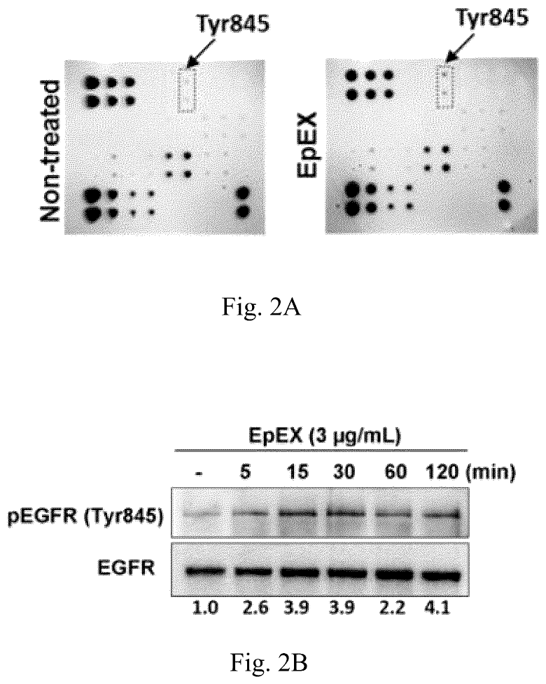

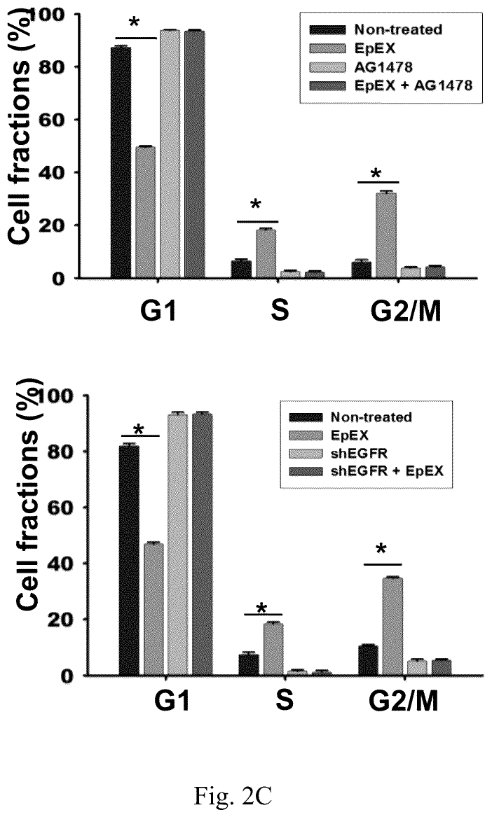

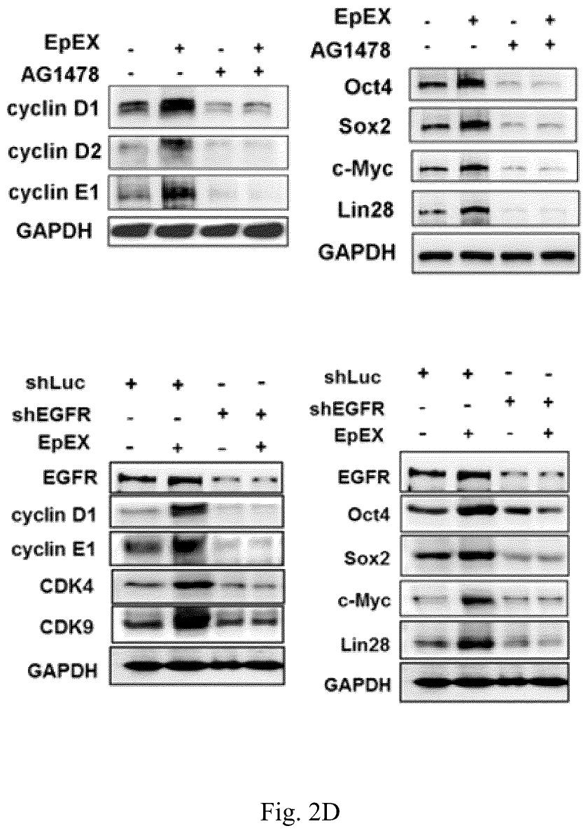

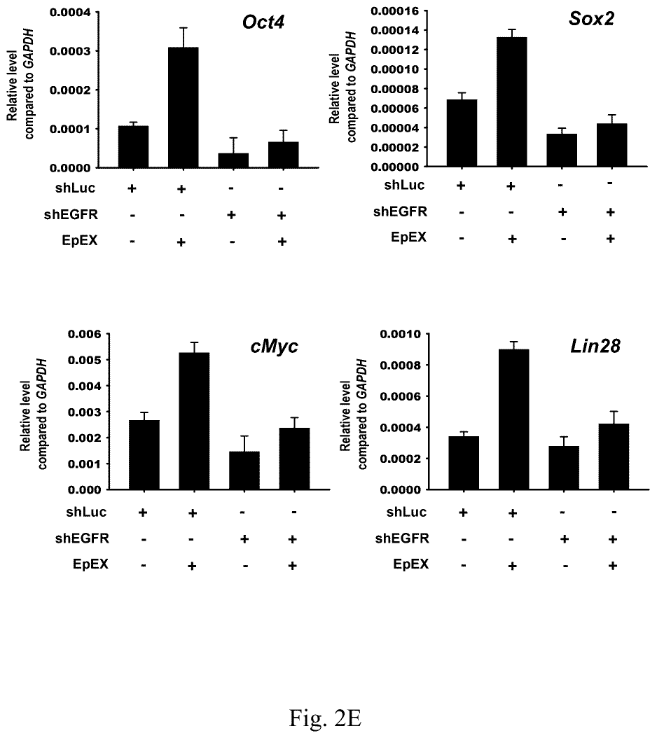

The EGF-EGFR signaling pathway has been shown to be critical for cell proliferation (Platt et al., 2009) and self-renewal in MSCs (Krampera et al., 2005; Tamama et al., 2006). Based on the knowledge that EpEX contains an EGF-like domain and activates EGFR signaling, as measured by a receptor kinase array (Kuan et al., 2017), we hypothesized that EpCAM/EpEX may serve as a cytokine or a growth factor to activate EGFR signaling and regulate cell growth and multipotency. Hence, we evaluated the phosphorylation state of EGFR by an EGFR membrane antibody array. We found that EpEX induced the phosphorylation of EGFR at Tyr845 (FIG. 2A). By Western blotting, we confirmed EpEX induced EGFR phosphorylation at Tyr845 in a time-dependent manner (FIG. 2B).

We also showed that both EGFR inhibitor (AG1478) and EGFR shRNA attenuated EpEX-induced cell cycle progression (FIG. 2C). By Western blotting, we showed that inhibition of EGFR by shRNA or inhibitor abolished EpEX-induced protein expression of cell cycle regulators, cyclin D1, cyclin D2, cyclin E1, CDK4 and CDK9, and pluripotency markers, Oct4, Sox2, c-Myc and Lin28 (FIG. 2D). By qPCR, we found that EpEX-induced increases in transcript levels of pluripotency markers, including Oct4, Sox2, c-Myc and Lin28, were also reversed by shEGFR (FIG. 2E). Taking these results together, we conclude that EpEX may induce cell proliferation and multipotency in MSCs through activation of EGFR.

### 2.3 EpEX Induces Cell Proliferation and Self-Renewal Via STAT3

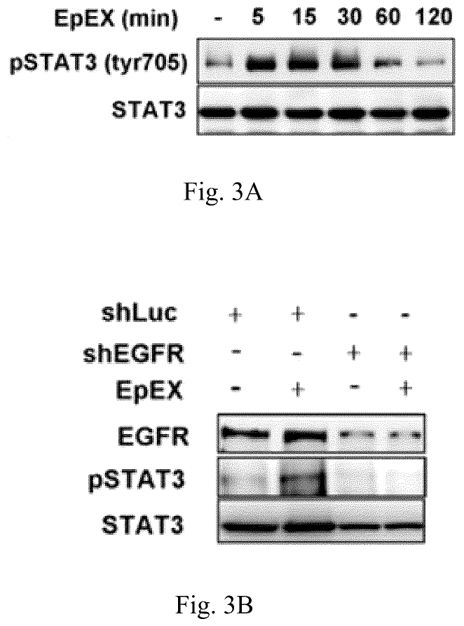

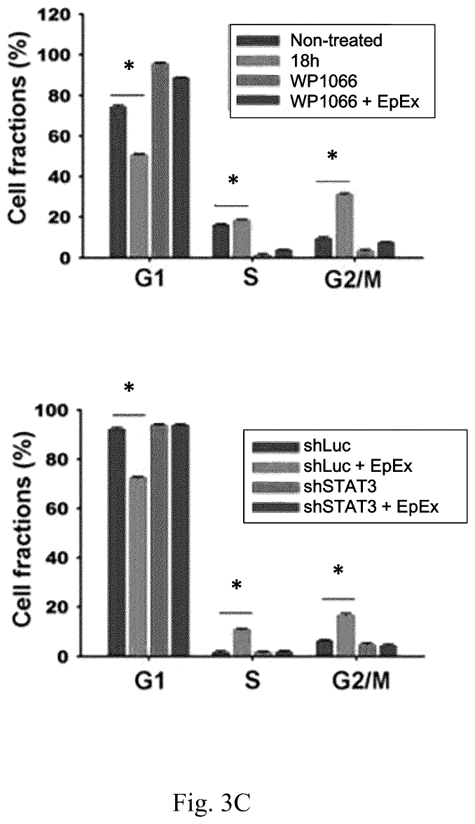

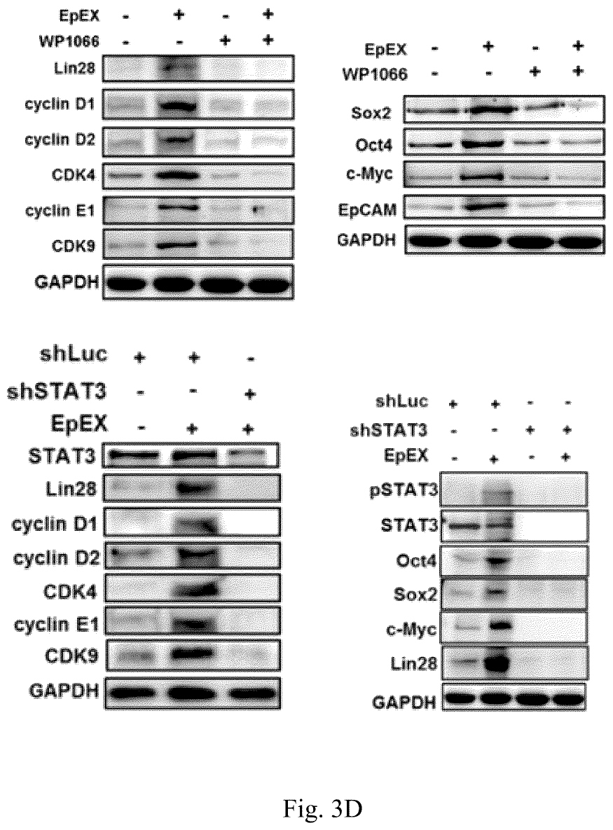

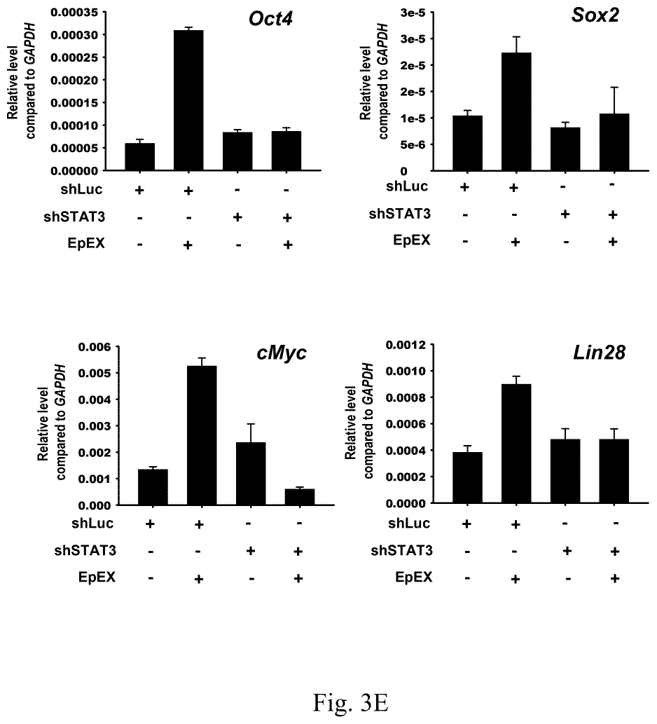

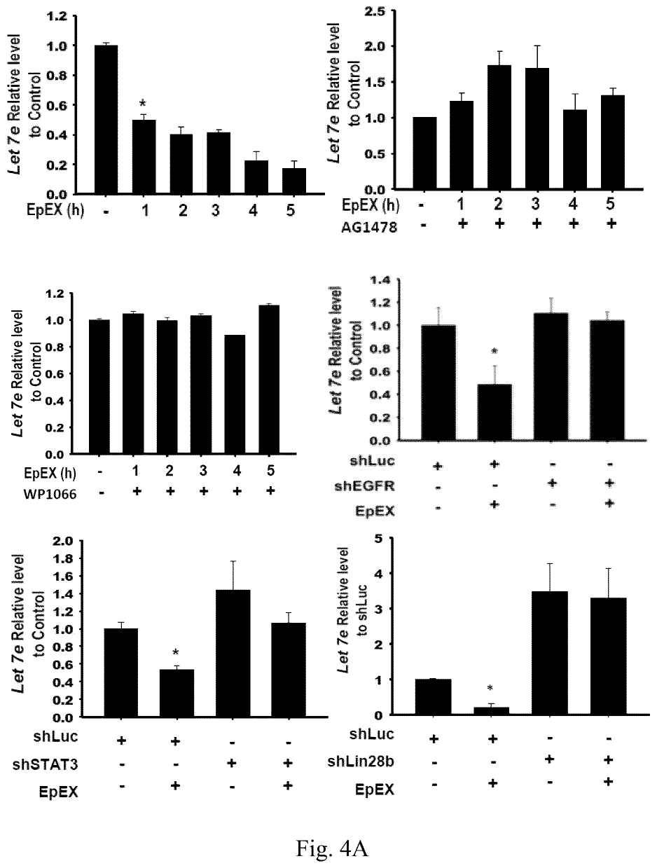

Previous studies have shown that STAT3 is a potent downstream effector of EGFR (Markovic and Chung, 2012; Song and Grandis, 2000) and also that STAT3 plays a crucial role in pluripotency maintenance (Raz et al., 1999). Furthermore, we have demonstrated that STAT3 signaling is essential for EpCAM/EpEX promotion of iPSC reprogramming (Kuan et al., 2017). Here we found that EpEX stimulates STAT3 phosphorylation shortly after treatment (FIG. 3A). Moreover, we found that EpEX-induced phosphorylation of STAT3 was abolished by EGFR knockdown, suggesting that EpEX induces STAT3 signaling through EGFR activation (FIG. 3B).

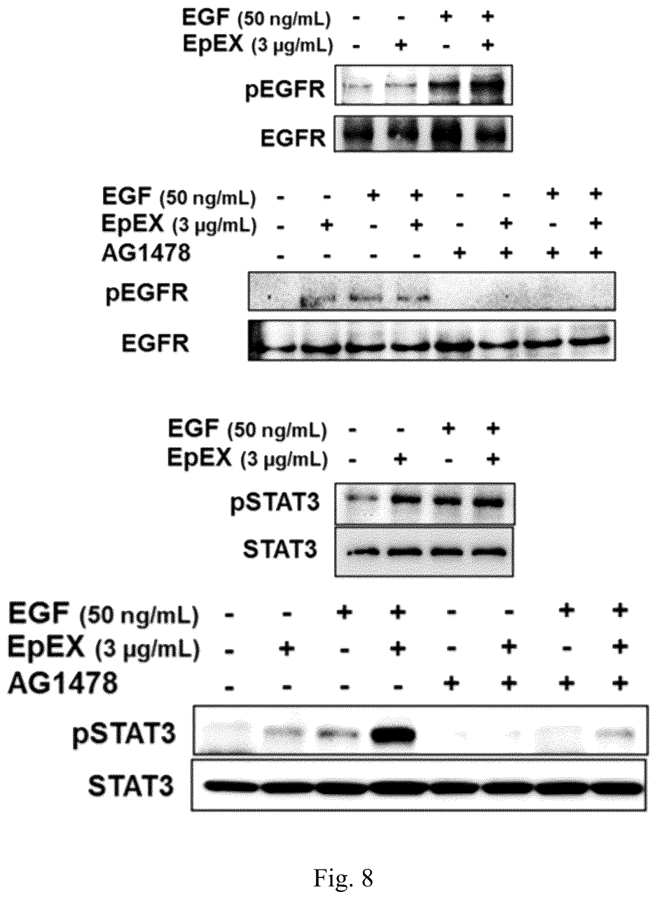

Because EGF is a cognate ligand for EGFR, we tested the effects of EGF on EGFR activation and STAT3 phosphorylation. Results showed that EpEX can induce EGFR phosphorylation as well as EGF activation. Moreover, we found that pretreatment of EGFR inhibitor, AG1478, can attenuate the activation of EGFR by either EGF or EpEX. Interestingly, we also confirmed that, similar to EpEX, EGF can induce the phosphorylation of STAT3 and that AG1478 can attenuate the activation of STAT3 by either EGF or EpEX. See FIG. 8.

We further investigated whether STAT3 signaling is involved in EpEX-induced cell growth and stemness of MSCs. By flow cytometry, we showed that STAT3 inhibitor (WP1066) and knockdown of STAT3 both attenuated EpEX-induced changes in cell cycle progression (FIG. 3C). By Western blotting, we also found that inhibition of STAT3 blocked EpEX-induced protein expression of cell cycle regulators, cyclin D1, cyclin D2, cyclin D3, cyclin E1, CDK4 and CDK9 and pluripotency markers, Oct4, Sox2, c-Myc and Lin28 (FIG. 3D). By qPCR, we also showed that inhibition of STAT3 prevented EpEX-increased gene expression of stemness markers, Oct4, Sox2, c-Myc and Lin28 (FIG. 3E). Furthermore, we also showed that knockdown of EpCAM decreased the level of phospho-STAT3, stemness markers and cell cycle regulators as well (FIG. 13).

### 2.4 EpEX Suppresses Let-7 Through EGFR-STAT3 Signaling

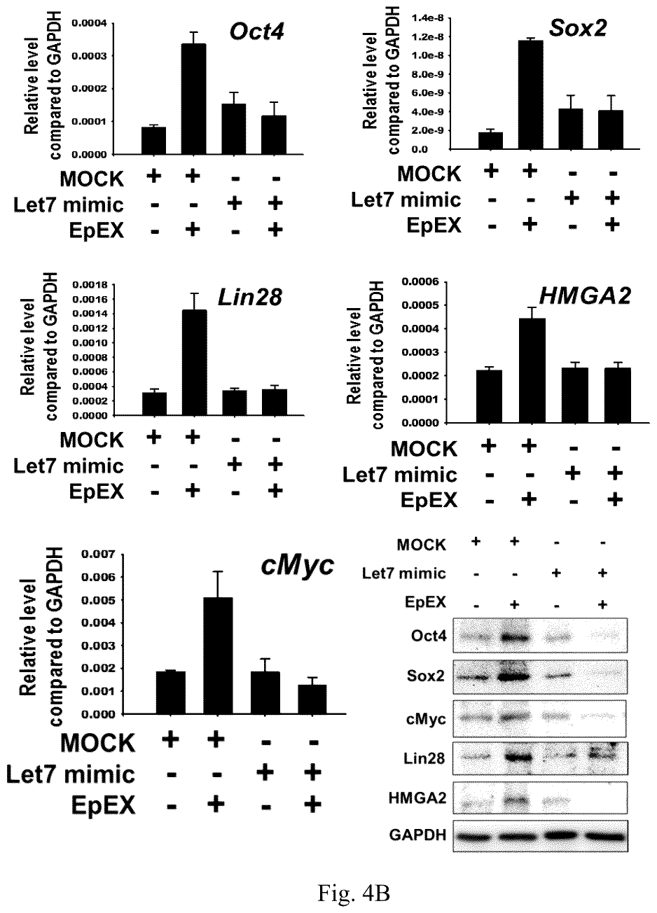

Previous studies have shown that Lin28 inhibits the miRNA, let-7, thereby increasing the levels of pluripotency factors (Lee et al., 2016; Piskounova et al., 2011; Stefani et al., 2015; Triboulet et al., 2015; Wang et al., 2015). Thus, we further examined if EpEX decreased the level of let-7. By qPCR, we showed that EpEX decreased the level of let-7 (FIG. 4A). We next tested whether EpEX-induced inhibition of let-7 expression occurs via STAT3 and EGFR and found that EpEX suppression of let-7 expression was attenuated by shEGFR, shSTAT3 or shLin28 (FIG. 4A). These results indicated that EGFR, STAT3 and Lin28 are necessary in EpEX regulation of let-7 expression.

Next, we used a let-7 mimetic to test whether let-7 suppression is necessary for EpEX-induced increase of pluripotency markers. We found that pretreatment with the let-7 mimetic abolished EpEX-induced gene and protein expression of Oct4, Sox2, c-Myc and Lin28 (FIG. 4B), confirming the importance of let-7 suppression in this process.

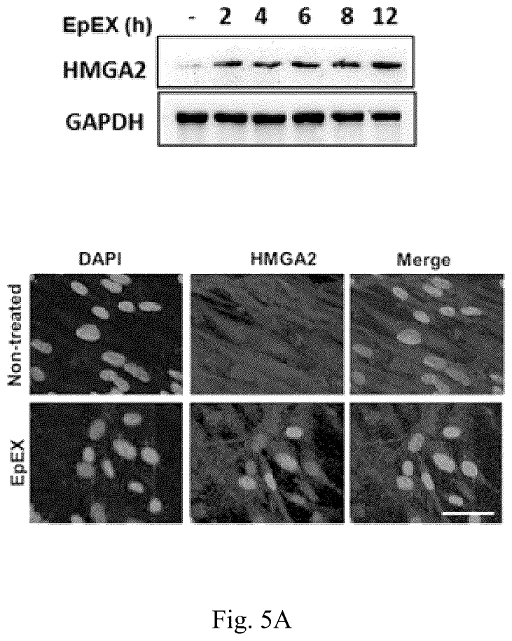

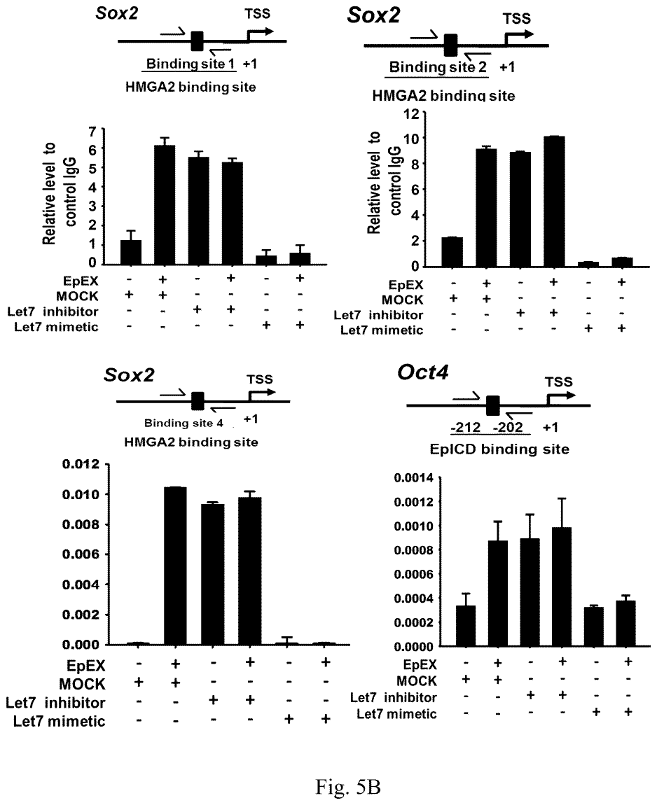

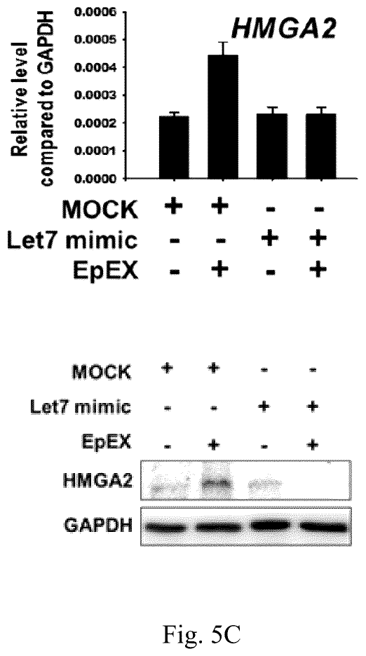

Similar to our findings, Lin28 was previously shown to decrease the level of let-7 (Piskounova et al., 2011), and furthermore, let-7 has been shown to suppress transcription of Oct4 and Sox2 through inhibition of transcription cofactors, AT-rich interaction domain molecule 3B (ARID3B) and high-mobility group AT-hook 2 (HMGA2) (Chien et al., 2015; Guo et al., 2006; Liao et al., 2016). The expression of HMGA2 is ubiquitous and abundant, and it has an important role during embryonic development (Monzen et al., 2008). Moreover, HMGA2 expression has been shown to promote stem cell self-renewal, while decreased expression is associated with stem cell aging (Li et al., 2007; Li et al., 2006; Nishino et al., 2008; Pfannkuche et. al., 2009). In normal adult tissues, the level of HMGA2 is very low, but the protein is highly expressed in many types of cancer cells, where it facilitates oncogene expression (Fusco and Fedele, 2007; Mahajan et al., 2010; Rawlinson et al., 2008; Wei et al., 2010). In addition, Lin28, which can suppress let-7 and upregulate expression of HMGA2, is important for self-renewal (Li et al., 2012) and maintenance of an undifferentiated state in cancer cells (Shell et al., 2007; Thornton and Gregory, 2012). Based on this information, we hypothesized that EpEX may also regulate HMGA2. Interestingly, we found that EpEX not only induced the level of HMGA2, but also induced its nuclear translocation by Western blotting (FIG. 5A) and immunofluorescent staining (FIG. 5B). Because HMGA2 belongs to the high mobility group with AT-hook DNA binding domain family of proteins, it changes DNA conformation by binding to AT-rich regions in the DNA and interacts with other transcription factors, rather than directly activating transcription itself (Cleynen and Van de Ven, 2008; Pfannkuchent al., 2009). Therefore, we asked whether EpEX treatment induces HMGA2 to bind to the promoters of pluripotency genes. By ChIP, we showed that EpEX can induce HMGA2 binding to the promoters of Oct4 and Sox2, while ablation of EGFR, STAT3 or Lin28 prevented the effect (data not shown). We further tested whether EpEX-induced HMGA2 binding is dependent on regulation of let-7. Pretreatment with let-7 mimetic abrogated the effect of EpEX, while treatment of the let-7 inhibitor was sufficient to induce the binding of HMGA2 to the promoters of Oct4 (FIG. 5B). In addition, we showed that the let-7 mimetic abolished EpEX-induced gene and protein expression of HMGA2 (FIG. 5C).

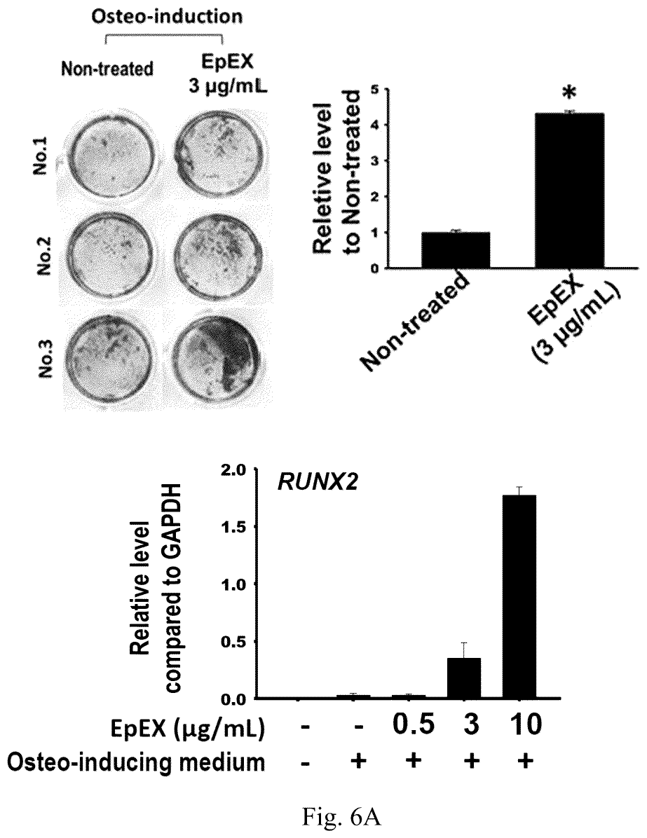

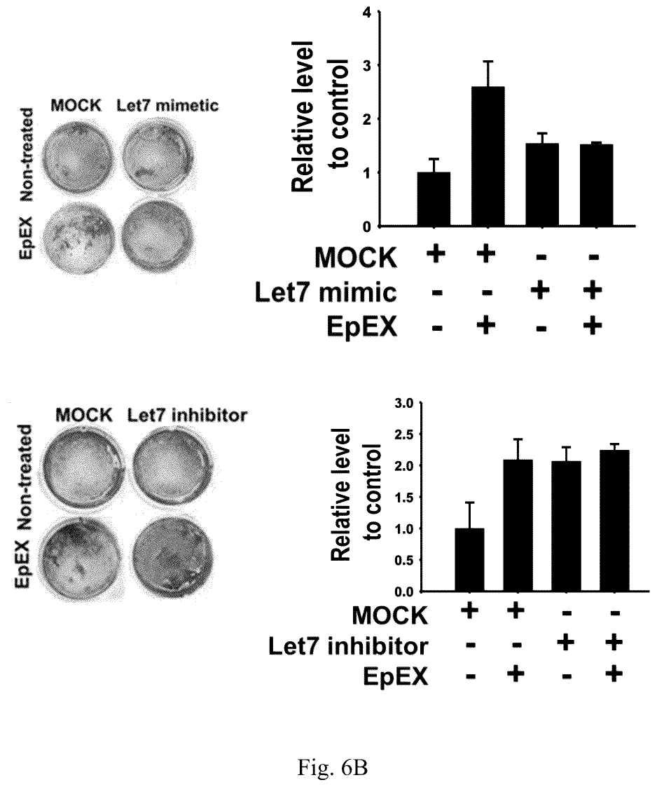

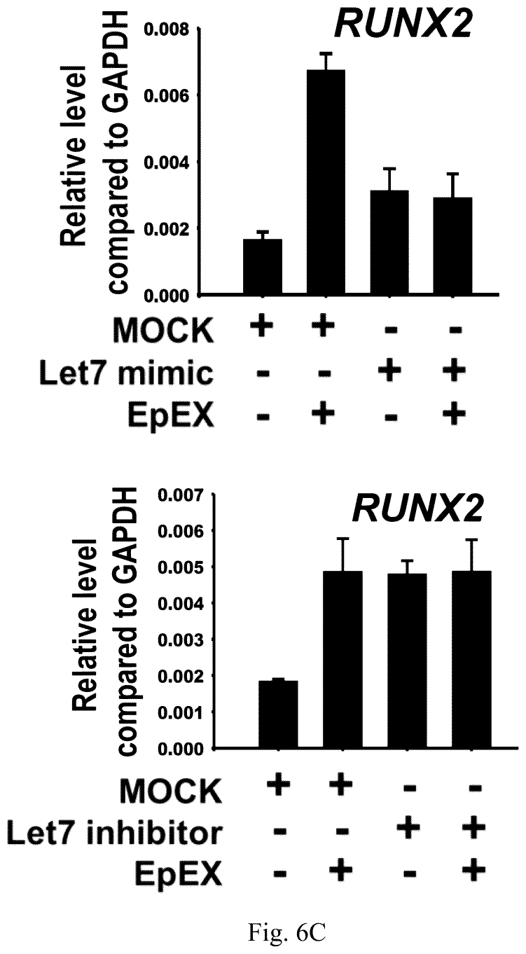

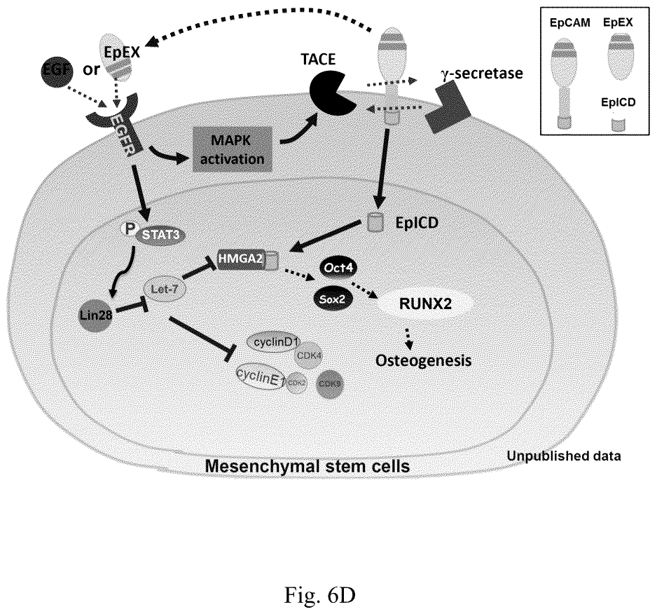

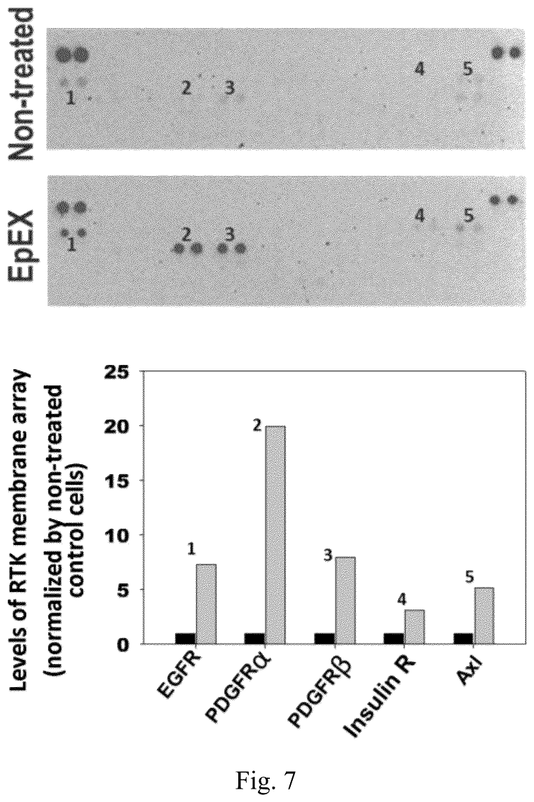

A previous study demonstrated that the upregulation of Oct4 and Sox2 can promote osteogenesis of MSCs (Matic et al., 2016). Therefore, we surmised that EpEX may also promote osteogenesis via upregulation of Oct4 and Sox2. To this end, we showed that the EpEX treatment during osteo-induction promotes osteogenesis by 4-fold when compared to controls (FIG. 6A). We also measured gene expression of the osteogenetic marker, RUNX2, and found that EpEX increased the transcript level (FIG. 6A). Next, we examined whether EpEX-enhanced osteogenesis is dependent on downregulation of let-7. We found that pretreatment of let-7 mimetic can abolish EpEX enhancements in osteogenesis, while let-7 inhibitor can increase osteogenesis (FIG. 6B). Finally, we showed that let-7 mimetic attenuated EpEX-induced gene expression of RUNX2 (FIG. 6C), while let-7 inhibitor increased RUNX2 gene expression (FIG. 6C).

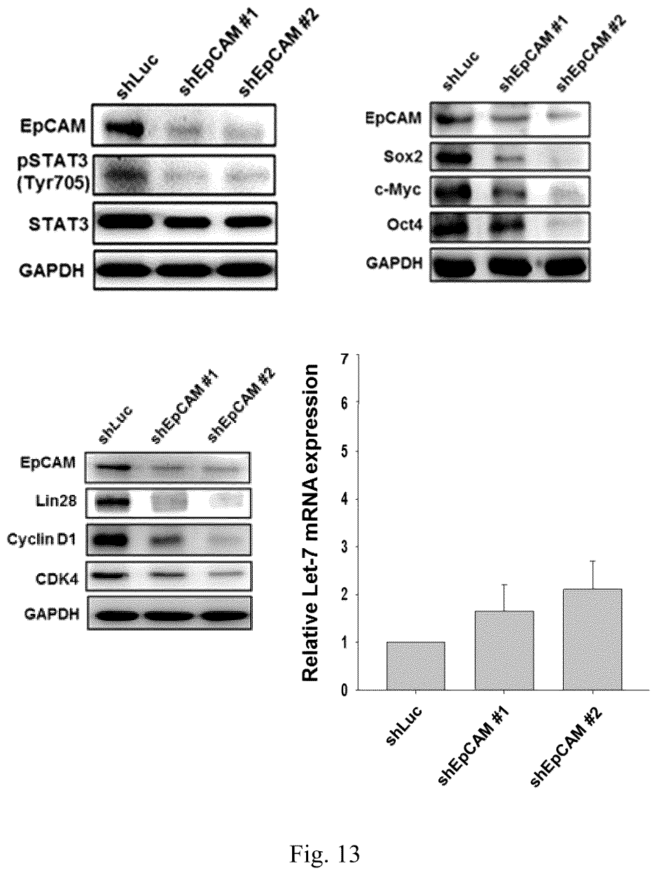

Our results showed that the treatment of EpEX can induce expression of the gene for Oct4, we examined the binding of EpICD to the Oct4 promoter by single ChIP and double ChIP assays. We pulled down EpICD and probed for a specific binding site on the Oct4 promoters and found that EpICD can indeed associate with the Oct4 promoter. We further investigated whether EpICD and HMGA2 could form a complex to bind to the promoter of Oct4. We sequentially pulled down EpICD and HMGA2, followed by probing of the binding site within the Oct4 promoter. The results showed that the EpICD-HMGA2 complex was associated with the Oct4 promotor. See FIG. 12.

### 2.5 EpEX Induced the Phosphorylation and Activity of TACE and γ-Secretase

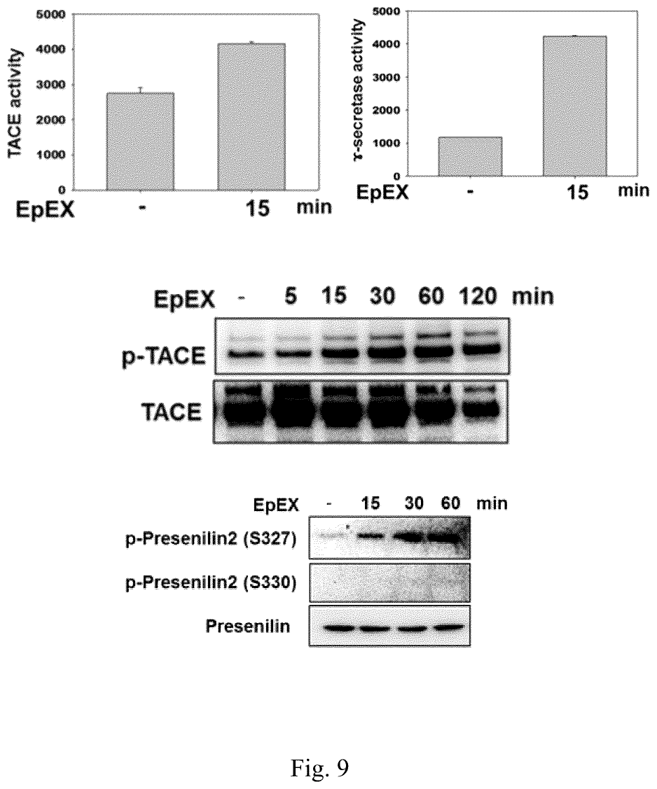

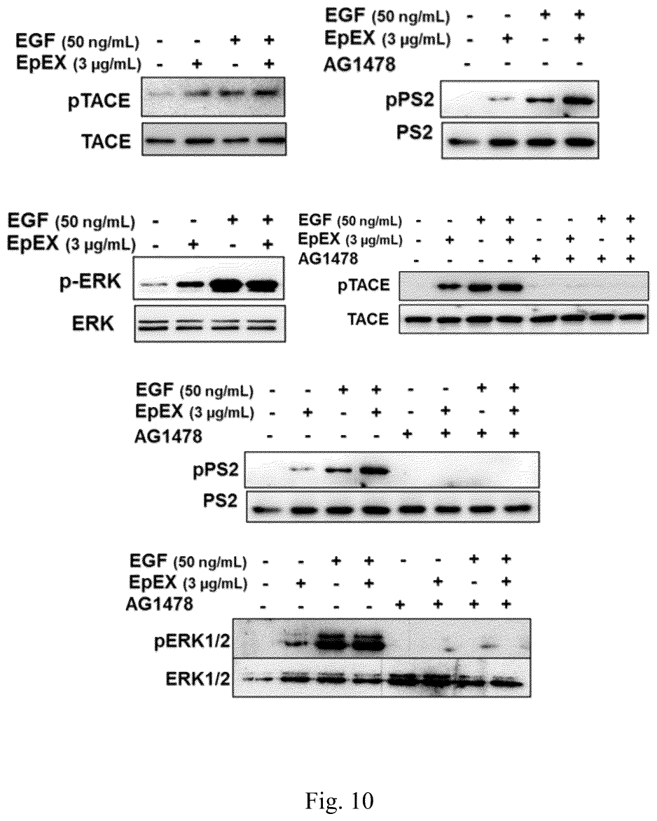

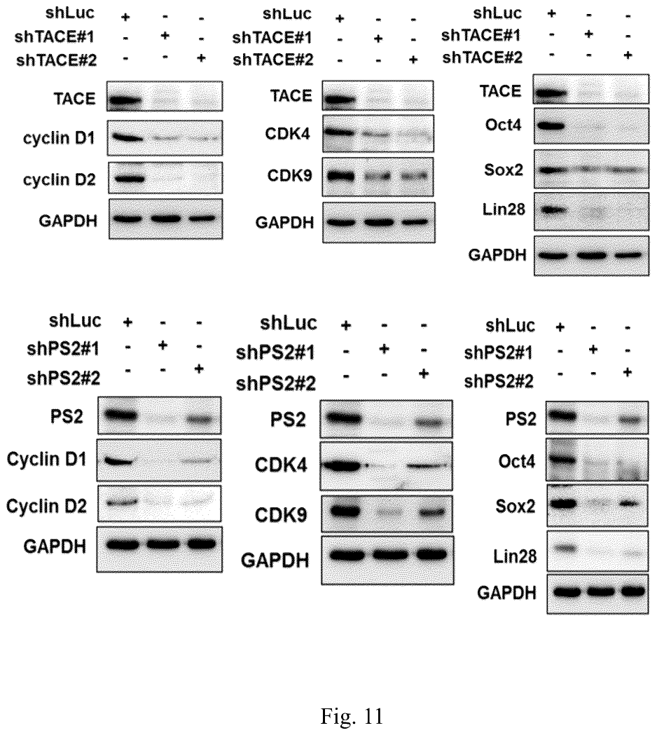

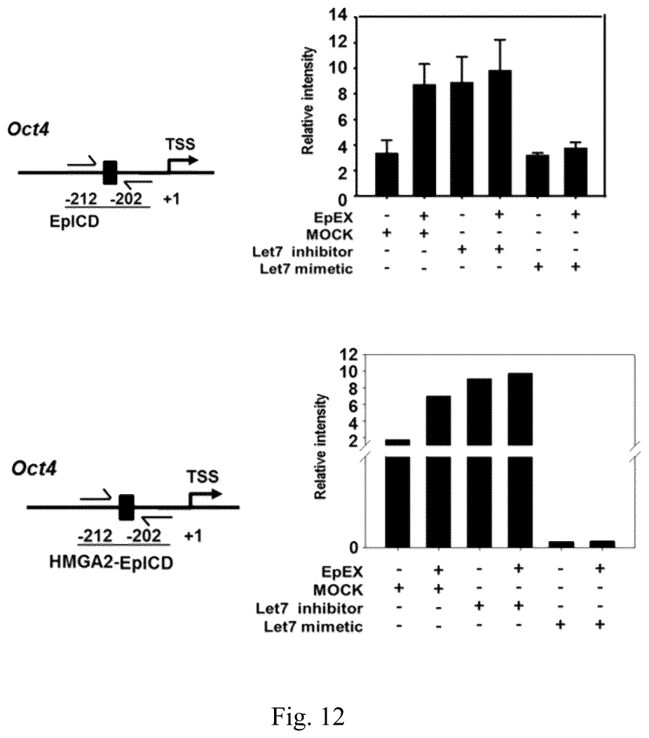

Previous studies indicate that EpCAM can be cleaved by the sheddase, TACE, leading to the release of soluble EpEX. This release may then trigger an autocrine cell signaling response (Maetzel et al., 2009). Because EpCAM signaling is processed both by TACE and γ-secretase, we investigated the effect of EpEX on TACE and γ-secretase activities. We detected the phosphorylation and activation of TACE and γ-secretase in EpEX-stimulated MSCs. The results of these assays showed that the activation of TACE and γ-secretase were induced by EpEX treatment in MSCs. We also showed the phosphorylation of TACE and γ-secretase were induced by EpEX. See FIG. 9. Next, we used an EGFR inhibitor to examine whether EpEX-induced activation of TACE and γ-secretase requires EGFR signaling. We showed that EpEX-induced phosphorylation of TACE and presenilin-2 can be abolished by the addition of EGFR inhibitor. We also investigated the upstream signaling that may result in activation of the TACE enzyme. ERK1/2 has been reported to regulate the activity of TACE, and we showed that EpEX can induce the EGFR-dependent phosphorylation of ERK1/2. See FIG. 10. Next we wanted to examine whether TACE and γ-secretase play roles in maintaining protein levels of cell cycle regulators and pluripotency factors. We found that knockdown of TACE or γ-secretase can inhibit the expression of cell cycle regulator and pluripotency markers. See FIG. 11.

### 3. Summary

The understanding of the mechanism for pluripotency has been greatly advanced through the discovery of induced pluripotent stem cells (iPSCs). However, the study of iPSCs for cell therapy is just the beginning; with many areas remain to be explored. Mesenchymal stem cells (MSCs) are widely considered to be an attractive cell source for novel regenerative therapies. However, the clinical application of MSCs depends on successful expansion in culture. Currently, maintenance of multipotency and self-renewal in cultured MSCs is especially challenging, because little is known about the cell-specific molecular mechanisms that regulate these processes. Hence, the development and mechanistic description of novel strategies to maintain or enhance multipotencyin MSCs will be vital to future clinical use. Here, we show that extracellular domain of EpCAM (EpEX) significantly enhances cell proliferation and increases the levels of pluripotency factorsthrough EGFR-STAT3-Lin28signaling in human bone marrow MSCs. Moreover, we found that EpEX-induced Lin28 can reduce let-7 miRNA expression, thereby upregulating the transcription factor, HMGA2, which activates transcription of pluripotency factors.

Surprisingly, we found that EpEX treatment also enhances osteogenesis of MSCs under differentiation conditions, as evidenced by increases in the osteogenetic marker, RUNX2. Taken together, our results describe a novel function of EpEX, which stimulates EGFR signaling toexert context-dependent effects on MSCs, promoting cell proliferationand multipotency under maintenance conditions and osteogenesis under differentiation conditions. We believe that our finding offer linkage between basic and medical research and will probably strengthen even more by the recent emergence of human induced pluripotent stem cells. MSCs are powerful tools for bridging the gap from our accumulated knowledge of regenerative medicine, as well as to a wide spectrum of medical and pharmaceutical research and development.

The MSCs from adults have limitations for clinical use due to narrow division capacity and limited survival; hence the maintenance of stemnessand development of MSCs is at present under intensive investigation. In the present study, we found that upon the stimulation of EpEX, EpEX induces the phosphorylation of EGFR-STAT3 signaling, and subsequently upregulates the level of Lin28 which inhibitslet7. The mechanisms lead to inhibition of let7 and thus increase a transcription factor, HMGA2, which can bind to the promoters of Oct4 and Sox2. The EpEX-increased Oct4 and Sox2 can promote the osteogenesis of MSCs during osteo-induction. Therefore, based on these evidences, we emphasize that we can increases the multipotency of MSCs by treatment of soluble EpEX protein and EpEX significantly enhances the osteogenic capacity of MSCsduring osteo-induction. We present not only that the extracellular domain of adhesion molecule can serve as a cytokine and has pleiotropic activity in MSC, but also offer a new strategy for enhancing cell proliferation and multipotency of MSCs.

Sequence Information

SEQUENCE ID NO: 1 (HUMAN Epithelial cell adhesion
molecule, full length)
(Underlined portion: extracellular domain, 1-265
a.a.)
(Bolded portion: transmembrane domain, 266-288
a.a.)
(double-underlined portion: intracellular domain,
289-314 a.a.)
MAPPQVLAFGLLLAAATATFAAAQEECVCENYKLAVNCFVNNNRQCQCTSV
GAQNTVICSKLAAKCLVMKAEMNGSKLGRRAKPEGALQNNDGLYDPDCDES
GLFKAKQCNGTSMCWCVNTAGVRRTDKDTEITCSERVRTYWIIIELKHKAR
EKPYDSKSLRTALQKEITTRYQLDPKFITSILYENNVITIDLVQNSSQKTQ
NDVDIADVAYYFEKDVKGESLFHSKKMDLTVNGEQLDLDPGQTLIYYVDEK
APEFSMQGLKAGVIAVIVVVVIAVVAGIVVLVISRKKRMAKYEKAEIKEMG
EMHRELNA
SEQUENCE ID NO: 2 (HUMAN Epithelial cell adhesion
molecule, extracellular domain)
MAPPQVLAFGLLLAAATATFAAAQEECVCENYKLAVNCFVNNNRQCQCTSV
GAQNTVICSKLAAKCLVMKAEMNGSKLGRRAKPEGALQNNDGLYDPDCDES
GLFKAKQCNGTSMCWCVNTAGVRRTDKDTEITCSERVRTYWIIIELKHKAR
EKPYDSKSLRTALQKEITTRYQLDPKFITSILYENNVITIDLVQNSSQKTQ
NDVDIADVAYYFEKDVKGESLFHSKKMDLTVNGEQLDLDPGQTLIYYVDEK
APEFSMQGLK
SEQUENCE ID NO: 3 (HUMAN Epithelial cell adhesion
molecule, transmembrane domain)
AGVIAVIVVVVIAVVAGIVVLVI
SEQUENCE ID NO: 4 (HUMAN Epithelial cell adhesion
molecule, intracellular domain)
SRKKRMAKYEKAEIKEMGEMHRELNA

### REFERENCE

1. Beltrami, A. P., Cesselli, D., Bergamin, N., Marcon, P., Rigo, S., Puppato, E., D'Aurizio, F., Verardo, R., Piazza, S., Pignatelli, A., et al. (2007). Multipotent cells can be generated in vitro from several adult human organs (heart, liver, and bone marrow). Blood 110, 3438-3446.
2. Boiani, M., and Scholer, H. R. (2005). Regulatory networks in embryo-derived pluripotent stem cells. Nat Rev Mol Cell Biol 6, 872-884.
3. Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
4. Briggs, R., and King, T. J. (1952). Transplantation of Living Nuclei From Blastula Cells into Enucleated Frogs' Eggs. Proc Natl Acad Sci USA 38, 455-463.
5. Brighton, C. T., and Hunt, R. M. (1997). Early histologic and ultrastructural changes in microvessels of periosteal callus. J Orthop Trauma 11, 244-253.
6. Chambers, I., and Tomlinson, S. R. (2009). The transcriptional foundation of pluripotency. Development 136, 2311-2322.
7. Cheng, L., Sung, M. T., Cossu-Rocca, P., Jones, T. D., MacLennan, G. T., De Jong, J., Lopez-Beltran, A., Montironi, R., and Looijenga, L. H. (2007). OCT4: biological functions and clinical applications as a marker of germ cell neoplasia. J Pathol 211, 1-9.
8. Chien, C. S., Wang, M. L., Chu, P. Y., Chang, Y. L., Liu, W. H., Yu, C. C., Lan, Y. T., Huang, P. I., Lee, Y. Y., Chen, Y. W., et al. (2015). Lin28B/Let-7 Regulates Expression of Oct4 and Sox2 and Reprograms Oral Squamous Cell Carcinoma Cells to a Stem-like State. Cancer Res 75, 2553-2565.
9. Cimadamore, F., Amador-Arjona, A., Chen, C., Huang, C. T., and Terskikh, A. V. (2013). SOX2-LIN28/let-7 pathway regulates proliferation and neurogenesis in neural precursors. Proc Natl Acad Sci USA 110, E3017-3026.
10. Cleynen, I., and Van de Ven, W. J. (2008). The HMGA proteins: a myriad of functions (Review). Int J Oncol 32, 289-305.
11. Dezawa, M., Ishikawa, H., Itokazu, Y., Yoshihara, T., Hoshino, M., Takeda, S., Ide, C., and Nabeshima, Y. (2005). Bone marrow stromal cells generate muscle cells and repair muscle degeneration. Science 309, 314-317.
12. Fang, X., Yoon, J. G., Li, L., Yu, W., Shao, J., Hua, D., Zheng, S., Hood, L., Goodlett, D. R., Foltz, G., et al. (2011). The SOX2 response program in glioblastoma multiforme: an integrated ChIP-seq, expression microarray, and microRNA analysis. BMC Genomics 12, 11.
13. Fusco, A., and Fedele, M. (2007). Roles of HMGA proteins in cancer. Nat Rev Cancer 7, 899-910.
14. Girouard, S. D., Laga, A. C., Mihm, M. C., Scolyer, R. A., Thompson, J. F., Zhan, Q., Widlund, H. R., Lee, C. W., and Murphy, G. F. (2012). SOX2 contributes to melanoma cell invasion. Lab Invest 92, 362-370.
15. Greber, B., Lehrach, H., and Adjaye, J. (2007). Silencing of core transcription factors in human EC cells highlights the importance of autocrine FGF signaling for self-renewal. BMC Dev Biol 7, 46.
16. Greco, S. J., Liu, K., and Rameshwar, P. (2007). Functional similarities among genes regulated by OCT4 in human mesenchymal and embryonic stem cells. Stem Cells 25, 3143-3154.
17. Guo, L., Chen, C., Shi, M., Wang, F., Chen, X., Diao, D., Hu, M., Yu, M., Qian, L., and Guo, N. (2013). Stat3-coordinated Lin-28-let-7-HMGA2 and miR-200-ZEB1 circuits initiate and maintain oncostatin M-driven epithelial-mesenchymal transition. Oncogene 32, 5272-5282.
18. Guo, Y., Chen, Y., Ito, H., Watanabe, A., Ge, X., Kodama, T., and Aburatani, H. (2006). Identification and characterization of lin-28 homolog B (LIN28B) in human hepatocellular carcinoma. Gene 384, 51-61.
19. Hattori, N., Nishino, K., Ko, Y. G., Hattori, N., Ohgane, J., Tanaka, S., and Shiota, K. (2004). Epigenetic control of mouse Oct-4 gene expression in embryonic stem cells and trophoblast stem cells. J Biol Chem 279, 17063-17069.
20. Huang, H. P., Chen, P. H., Yu, C. Y., Chuang, C. Y., Stone, L., Hsiao, W. C., Li, C. L., Tsai, S. C., Chen, K. Y., Chen, H. F., et al. (2011). Epithelial cell adhesion molecule (EpCAM) complex proteins promote transcription factor-mediated pluripotency reprogramming. J Biol Chem 286, 33520-33532.
21. Kerkis, I., Kerkis, A., Dozortsev, D., Stukart-Parsons, G. C., Gomes Massironi, S. M., Pereira, L. V., Caplan, A. I., and Cerruti, H. F. (2006). Isolation and characterization of a population of immature dental pulp stem cells expressing OCT-4 and other embryonic stem cell markers. Cells Tissues Organs 184, 105-116.
22. Kim, J. H., Jee, M. K., Lee, S. Y., Han, T. H., Kim, B. S., Kang, K. S., and Kang, S. K. (2009). Regulation of adipose tissue stromal cells behaviors by endogenic Oct4 expression control. PLoS One 4, e7166.
23. Kuan, II, Liang, K. H., Wang, Y. P., Kuo, T. W., Meir, Y. J., Wu, S. C., Yang, S. C., Lu, J., and Wu, H. C. (2017). EpEX/EpCAM and Oct4 or Klf4 alone are sufficient to generate induced pluripotent stem cells through STAT3 and HIF2alpha. Sci Rep 7, 41852.
24. Lee, H., Han, S., Kwon, C. S., and Lee, D. (2016). Biogenesis and regulation of the let-7 miRNAs and their functional implications. Protein Cell 7, 100-113.
25. Lengler, J., Bittner, T., Munster, D., Gawad Ael, D., and Graw, J. (2005). Agonistic and antagonistic action of AP2, Msx2, Pax6, Prox1 AND Six3 in the regulation of Sox2 expression. Ophthalmic Res 37, 301-309.
26. Lengner, C. J., Welstead, G. G., and Jaenisch, R. (2008). The pluripotency regulator Oct4: a role in somatic stem cells? Cell Cycle 7, 725-728.
27. Li, O., Li, J., and Droge, P. (2007). DNA architectural factor and proto-oncogene HMGA2 regulates key developmental genes in pluripotent human embryonic stem cells. FEBS Lett 581, 3533-3537.
28. Li, O., Vasudevan, D., Davey, C. A., and Droge, P. (2006). High-level expression of DNA architectural factor HMGA2 and its association with nucleosomes in human embryonic stem cells. Genesis 44, 523-529.
29. Li, Z., Gilbert, J. A., Zhang, Y., Zhang, M., Qiu, Q., Ramanujan, K., Shavlakadze, T., Eash, J. K., Scaramozza, A., Goddeeris, M. M., et al. (2012). An HMGA2-IGF2BP2 axis regulates myoblast proliferation and myogenesis. Dev Cell 23, 1176-1188.
30. Liao, T. T., Hsu, W. H., Ho, C. H., Hwang, W. L., Lan, H. Y., Lo, T., Chang, C. C., Tai, S. K., and Yang, M. H. (2016). let-7 Modulates Chromatin Configuration and Target Gene Repression through Regulation of the ARID3B Complex. Cell Rep 14, 520-533.
31. Lin, C. W., Liao, M. Y., Lin, W. W., Wang, Y. P., Lu, T. Y., and Wu, H. C. (2012). Epithelial cell adhesion molecule regulates tumor initiation and tumorigenesis via activating reprogramming factors and epithelial-mesenchymal transition gene expression in colon cancer. J Biol Chem 287, 39449-39459.
32. Lin, G., Garcia, M., Ning, H., Banie, L., Guo, Y. L., Lue, T. F., and Lin, C. S. (2008). Defining stem and progenitor cells within adipose tissue. Stem Cells Dev 17, 1053-1063.
33. Litvinov, S. V., Velders, M. P., Bakker, H. A., Fleuren, G. J., and Warnaar, S. O. (1994). Ep-CAM: a human epithelial antigen is a homophilic cell-cell adhesion molecule. J Cell Biol 125, 437-446.
34. Liu, K., Lin, B., Zhao, M., Yang, X., Chen, M., Gao, A., Liu, F., Que, J., and Lan, X. (2013a). The multiple roles for Sox2 in stem cell maintenance and tumorigenesis. Cell Signal 25, 1264-1271.
35. Liu, Y., Li, H., Feng, J., Cui, X., Huang, W., Li, Y., Su, F., Liu, Q., Zhu, J., Lv, X., et al. (2013b). Lin28 induces epithelial-to-mesenchymal transition and stemness via downregulation of let-7a in breast cancer cells. PLOS One 8, e83083.
36. Looijenga, L. H., Stoop, H., de Leeuw, H. P., de Gouveia Brazao, C. A., Gillis, A. J., van Roozendaal, K. E., van Zoelen, E. J., Weber, R. F., Wolffenbuttel, K. P., van Dekken, H., et al. (2003). POU5F1 (OCT3/4) identifies cells with pluripotent potential in human germ cell tumors. Cancer Res 63, 2244-2250.
37. Lu, T. Y., Lu, R. M., Liao, M. Y., Yu, J., Chung, C. H., Kao, C. F., and Wu, H. C. (2010). Epithelial cell adhesion molecule regulation is associated with the maintenance of the undifferentiated phenotype of human embryonic stem cells. Journal of Biological Chemistry 285, 8719-8732.
38. Maetzel, D., Denzel, S., Mack, B., Canis, M., Went, P., Benk, M., Kieu, C., Papior, P., Baeuerle, P. A., Munz, M., et al. (2009). Nuclear signalling by tumour-associated antigen EpCAM. Nat Cell Biol 11, 162-171.
39. Mahajan, A., Liu, Z., Gellert, L., Zou, X., Yang, G., Lee, P., Yang, X., and Wei, J. J. (2010). HMGA2: a biomarker significantly overexpressed in high-grade ovarian serous carcinoma. Mod Pathol 23, 673-681.
40. Masui, S., Nakatake, Y., Toyooka, Y., Shimosato, D., Yagi, R., Takahashi, K., Okochi, H., Okuda, A., Matoba, R., Sharov, A. A., et al. (2007). Pluripotency governed by Sox2 via regulation of Oct3/4 expression in mouse embryonic stem cells. Nat Cell Biol 9, 625-635.
41. Matic, I., Antunovic, M., Brkic, S., Josipovic, P., Mihalic, K. C., Karlak, I., Ivkovic, A., and Marijanovic, I. (2016). Expression of OCT-4 and SOX-2 in Bone Marrow-Derived Human Mesenchymal Stem Cells during Osteogenic Differentiation. Open Access Maced J Med Sci 4, 9-16.
42. Monk, M., and Holding, C. (2001). Human embryonic genes re-expressed in cancer cells. Oncogene 20, 8085-8091.
43. Monzen, K., Ito, Y., Naito, A. T., Kasai, H., Hiroi, Y., Hayashi, D., Shiojima, I., Yamazaki, T., Miyazono, K., Asashima, M., et al. (2008). A crucial role of a high mobility group protein HMGA2 in cardiogenesis. Nat Cell Biol 10, 567-574.
44. Nishino, J., Kim, I., Chada, K., and Morrison, S. J. (2008). Hmga2 promotes neural stem cell self-renewal in young but not old mice by reducing p16Ink4a and p19Arf Expression. Cell 135, 227-239.
45. Niwa, H., Miyazaki, J., and Smith, A. G. (2000). Quantitative expression of Oct-3/4 defines differentiation, dedifferentiation or self-renewal of ES cells. Nat Genet 24, 372-376.
46. Palmieri, S. L., Peter, W., Hess, H., and Scholer, H. R. (1994). Oct-4 transcription factor is differentially expressed in the mouse embryo during establishment of the first two extraembryonic cell lineages involved in implantation. Dev Biol 166, 259-267.
47. Pan, G., and Thomson, J. A. (2007). Nanog and transcriptional networks in embryonic stem cell pluripotency. Cell Res 17, 42-49.
48. Peng, G. H., and Chen, S. (2013). Double chromatin immunoprecipitation: analysis of target co-occupancy of retinal transcription factors. Methods Mol Biol 935, 311-328.
49. Pesce, M., and Scholer, H. R. (2001). Oct-4: gatekeeper in the beginnings of mammalian development. Stem Cells 19, 271-278.
50. Pfannkuche, K., Summer, H., Li, O., Hescheler, J., and Droge, P. (2009). The high mobility group protein HMGA2: a co-regulator of chromatin structure and pluripotency in stem cells? Stem Cell Rev 5, 224-230.
51. Pierantozzi, E., Gava, B., Manini, I., Roviello, F., Marotta, G., Chiavarelli, M., and Sorrentino, V. (2011). Pluripotency regulators in human mesenchymal stem cells: expression of NANOG but not of OCT-4 and SOX-2. Stem Cells Dev 20, 915-923.
52. Piskounova, E., Polytarchou, C., Thornton, J. E., LaPierre, R. J., Pothoulakis, C., Hagan, J. P., Iliopoulos, D., and Gregory, R. I. (2011). Lin28A and Lin28B inhibit let-7 microRNA biogenesis by distinct mechanisms. Cell 147, 1066-1079.
53. Pittenger, M. F., Mackay, A. M., Beck, S. C., Jaiswal, R. K., Douglas, R., Mosca, J. D., Moorman, M. A., Simonetti, D. W., Craig, S., and Marshak, D. R. (1999). Multilineage potential of adult human mesenchymal stem cells. Science 284, 143-147.
54. Rawlinson, N.J., West, W. W., Nelson, M., and Bridge, J. A. (2008). Aggressive angiomyxoma with t (12; 21) and HMGA2 rearrangement: report of a case and review of the literature. Cancer Genet Cytogenet 181, 119-124.
55. Reim, G., and Brand, M. (2002). Spiel-ohne-grenzen/pou2 mediates regional competence to respond to Fgf8 during zebrafish early neural development. Development 129, 917-933.
56. Shell, S., Park, S. M., Radjabi, A. R., Schickel, R., Kistner, E. O., Jewell, D. A., Feig, C., Lengyel, E., and Peter, M. E. (2007). Let-7 expression defines two differentiation stages of cancer. Proc Natl Acad Sci USA 104, 11400-11405.
57. Sperger, J. M., Chen, X., Draper, J. S., Antosiewicz, J. E., Chon, C. H., Jones, S. B., Brooks, J. D., Andrews, P. W., Brown, P. O., and Thomson, J. A. (2003). Gene expression patterns in human embryonic stem cells and human pluripotent germ cell tumors. Proc Natl Acad Sci USA 100, 13350-13355.
58. Stefani, G., Chen, X., Zhao, H., and Slack, F. J. (2015). A novel mechanism of LIN-28 regulation of let-7 microRNA expression revealed by in vivo HITS-CLIP in C. elegans. RNA 21, 985-996.
59. Stenderup, K., Justesen, J., Clausen, C., and Kassem, M. (2003). Aging is associated with decreased maximal life span and accelerated senescence of bone marrow stromal cells. Bone 33, 919-926.
60. Stolzing, A., Jones, E., McGonagle, D., and Scutt, A. (2008). Age-related changes in human bone marrow-derived mesenchymal stem cells: consequences for cell therapies. Mech Ageing Dev 129, 163-173.
61. Tai, M. H., Chang, C. C., Kiupel, M., Webster, J. D., Olson, L. K., and Trosko, J. E. (2005). Oct4 expression in adult human stem cells: evidence in support of the stem cell theory of carcinogenesis. Carcinogenesis 26, 495-502.
62. Takahashi, K., Mitsui, K., and Yamanaka, S. (2003). Role of ERas in promoting tumour-like properties in mouse embryonic stem cells. Nature 423, 541-545.
63. Takahashi, K., and Yamanaka, S. (2006). Induction of pluripotent stem cells from mouse embryonic and adult fibroblast cultures by defined factors. Cell 126, 663-676.
64. Taniguchi, N., Carames, B., Hsu, E., Cherqui, S., Kawakami, Y., and Lotz, M. (2011). Expression patterns and function of chromatin protein HMGB2 during mesenchymal stem cell differentiation. J Biol Chem 286, 41489-41498.
65. Thornton, J. E., and Gregory, R. I. (2012). How does Lin28 let-7 control development and disease? Trends Cell Biol 22, 474-482.
66. Triboulet, R., Pirouz, M., and Gregory, R. I. (2015). A Single Let-7 MicroRNA Bypasses LIN28-Mediated Repression. Cell Rep 13, 260-266.
67. Valero, M. C., Huntsman, H. D., Liu, J., Zou, K., and Boppart, M. D. (2012). Eccentric exercise facilitates mesenchymal stem cell appearance in skeletal muscle. PLOS One 7, e29760.
68. Wang, T., Wang, G., Hao, D., Liu, X., Wang, D., Ning, N., and Li, X. (2015). Aberrant regulation of the LIN28A/LIN28B and let-7 loop in human malignant tumors and its effects on the hallmarks of cancer. Mol Cancer 14, 125.
69. Wei, J. J., Wu, J., Luan, C., Yeldandi, A., Lee, P., Keh, P., and Liu, J. (2010). HMGA2: a potential biomarker complement to P53 for detection of early-stage high-grade papillary serous carcinoma in fallopian tubes. Am J Surg Pathol 34, 18-26.
70. Xu, R. H., Sampsell-Barron, T. L., Gu, F., Root, S., Peck, R. M., Pan, G., Yu, J., Antosiewicz-Bourget, J., Tian, S., Stewart, R., et al. (2008). NANOG is a direct target of TGFbeta/activin-mediated SMAD signaling in human ESCs. Cell Stem Cell 3, 196-206.
71. Yeom, S. Y., Nam, D. H., and Park, C. (2014). RRAD promotes EGFR-mediated STAT3 activation and induces temozolomide resistance of malignant glioblastoma. Mol Cancer Ther 13, 3049-3061.
72. Zhou, S., Greenberger, J. S., Epperly, M. W., Goff, J. P., Adler, C., Leboff, M. S., and Glowacki, J. (2008). Age-related intrinsic changes in human bone-marrow-derived mesenchymal stem cells and their differentiation to osteoblasts. Aging Cell 7, 335-343.

What is claimed is:

1. A method for culturing mesenchymal stem cells (MSCs), comprising culturing the MSCs under a condition in the presence of an isolated EpCAM polypeptide.

2. The method of claim 1, wherein the EpCAM polypeptide is an extracellular domain of EpCAM.

3. The method of claim 1, wherein the extracellular domain of EpCAM comprises SEQ ID NO: 2 or an amino acid sequence at least 90% identical to SEQ ID No: 2.

4. The method of claim 1, wherein the EpCAM polypeptide is present in an amount effective in promoting expansion and/or multipotency of the MSCs.

5. The method of claim 1, wherein the MSCs are cultured in a medium composition where the EpCAM polypeptide is present in an amount of 1-50 μg/mL.

6. The method of claim 1, wherein the medium composition comprises (i) Dulbecco's modified Eagle's medium-low glucose (DMEM-LG) supplemented with 0.1-5 mM glutamine, 5% to 25% FBS, and 1-50 μg/mL EpCAM polypeptide, or (ii) Dulbecco's modified Eagle's medium-high glucose (DMEM-HG) supplemented with 5% to 25% FBS, 0.05-1 μM dexamethasone, 1-50 mM β-glycerophosphate, 0.01-0.1 mM ascorbic acid-phosphate and 1-50 μg/mL EpCAM polypeptide.

7. The method of claim 1, wherein the EpCAM polypeptide is present in an amount effective in promoting expansion and multipotency of the MSCs.

8. A method for enhancing osteogenesis of mesenchymal stem cells (MSCs), comprising culturing the MSCs in an osteogenic induction medium which comprises one or more components for osteogenic induction selected from the group consisting of β-glycerophosphate, ascorbic acid, dexamethasone and any combination thereof, wherein the medium further comprises an isolated EpCAM polypeptide.

9. The method of claim 8, wherein the EpCAM polypeptide is present in an amount effective in promoting expansion and/or multipotency of the MSCs.

10. The method of claim 8, wherein the EpCAM polypeptide is present in an amount effective in promoting expansion and multipotency of the MSCs.
